(19) United States
(12) Patent Application Publication
(10) Pub. No.: US 2026/0137326 A1
(43) Pub. Date: May 21, 2026

(54) MANAGING SIMULATED MAGNETOCARDIOGRAPHY (MCG) DATA

(71) Applicant: SB Technology, Inc., Tarrytown, NY (US)

(72) Inventors: Brian K. Cottle, Logan, UT (US); Geoffrey Iwata, San Francisco, CA (US); Kirstin Aschbacher, San Francisco, CA (US)

(21) Appl. No.: 19/385,054

(22) Filed: Nov. 10, 2025

Related U.S. Application Data
(60) Provisional application No. 63/723,576, filed on Nov. 21, 2024.
(60) Provisional application No. 63/754,307, filed on Feb. 5, 2025.

(51) Int. Cl. A61B 5/319

(52) U.S. Cl. CPC A61B 5/319

(57) ABSTRACT

The present disclosure relates to methods, systems, and apparatus, including computer programs encoded on computer storage media, for simulating magnetic data. An example method includes obtaining electrical data of an organ of a subject. The method further includes generating current data corresponding to a current source based on the electrical data of the organ of the subject. The method further includes generating simulated magnetic data of the organ of the subject based on the current data corresponding to the current source. The method further includes taking an action based on the simulated magnetic data.

### CROSS-REFERENCE TO RELATED APPLICATION

This application claims priority to U.S. Provisional Patent Application No. 63/723,576, entitled “Managing Simulated Magnetocardiography (MCG) Data” and filed on Nov. 21, 2024, the entire contents of which is incorporated herein by reference.

### TECHNICAL FIELD

The present disclosure relates generally medical imaging and artificial intelligence (AI).

### BACKGROUND

Measurements of bio-magnetic fields in a human subject can be used to understand potential disease states. For example, magnetoencephalography (MEG) is a noninvasive technique for investigating human brain activity. Magnetocardiography (MCG) is a non-invasive technique for measuring magnetic fields produced by electric currents in the heart. Similar to an electrocardiogram, a magneto-cardiogram has morphological features such as a QRS complex, P-waves, and T-waves.

### SUMMARY

The present disclosure describes technologies for simulating magnetic data.

In general, one innovative aspect of the subject matter described in this present disclosure can be embodied in methods that include the actions of obtaining, by one or more processors, electrical data of an organ of a subject. The actions include generating current data corresponding to a current source based on the electrical data of the organ of the subject. The actions further include generating simulated magnetic data of the organ of the subject based on the current data corresponding to the current source. The actions further include taking an action based on the simulated magnetic data.

Another aspect of the subject matter described in this present disclosure can be embodied in methods that include the actions of receiving, by one or more processors, vectorcardiography (VCG) data for a subject. The actions include generating current data corresponding to a current source based on the vectorcardiography (VCG) data. The actions further include generating simulated magnetocardiography (MCG) data for the subject based on the current data corresponding to the current source. The actions further include taking an action based on the simulated magnetocardiography (MCG) data.

Other embodiments of these aspects include corresponding computer systems, apparatus, and computer programs recorded on one or more computer storage devices, each configured to perform the actions of the methods. A system of one or more computers to be configured to perform particular operations or actions means that the system has installed software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

In some implementations, the current source is independent from a physiological geometry of the organ of the subject.

In some implementations, the current source is derived by approximating an electrical current of the organ of the subject as a set of current dipoles. An electrical activity of the organ at a single time point is represented by a corresponding single current dipole.

In some implementations, the organ is a heart, and the current data includes vector data that represents electrical current data of the heart. The method further includes generating a set of vector data for each of a plurality of heart beats of the subject.

In some implementations, the current data includes information of an average direction of a current flowing in the organ of the subject at a given point in time.

In some implementations, the current source includes a single current dipole.

In some implementations, the current source includes a series of current dipoles, and the simulated magnetic data of the organ of the subject is associated with a synthetic result of magnetic fields from the series of current dipoles of the current source.

In some implementations, generating the simulated magnetic data of the organ of the subject based on the current data includes generating representative data of a magnetic field of the organ of the subject based on the current data by physics-based forward modeling. The simulated magnetic data includes the representative data of the magnetic field.

In some implementations, generating the simulated magnetic data of the organ of the subject based on the current data includes converting the current data into the simulated magnetic data using a Biot-Savart equation by integrating the current data along a path C of the current source according to the Biot-Savart equation expressed as B(r)=μ₀/4π∫_{C} Idl×r′/|r′|³, where μ₀ is permeability of free space, r represents a radius away from the current source, B(r) is a magnetic field generated by a given current at radius r away from the current source, dl represents an infinitesimal section of the current source, I is a current magnitude of that section of the current source, C is the path of the current source.

In some implementations, obtaining the electrical data of the organ of the subject includes transforming measured current data of the organ of the subject into the electrical data using a transformation approach. The transformation approach includes at least one of Kors Regression, Kors quasi-orthogonal, inverse Dower, or a linear regression-based transformation. In certain embodiments, the transformation approach includes a non-linear transformation. For example, the non-linear transformation can be performed using a transformation machine learning (ML) model that includes a neural network.

In some implementations, generating the current data corresponding to the current source based on the electrical data of the organ of the subject includes: for each single time point of a plurality of sample time points corresponding to the electric data, extracting corresponding vector amplitudes along axes of a coordinate system of a vector representing the electric data at the single time point; and calculating an electric field value at a given sensor location relative to the current source in the coordinate system. The current data includes a plurality of electric field values respectively calculated at the plurality of sample time points.

In some implementations, the electrical data includes vectorcardiography (VCG) data, and the simulated magnetic data includes MCG data. The method includes receiving electrocardiography (ECG) data of the organ of the subject and converting the ECG data to VCG data.

In some implementations, the electrical data includes 12-lead ECG measurement data, the 12-lead ECG measured data includes 1×12 matrix data, and the current source includes 1×3 matrix data.

In some implementations, the simulated magnetic data includes data corresponding to a respective magnetic field at a position of each of a plurality of simulated sensors near or around the current source.

In some implementations, generating the simulated magnetic data of the organ of the subject includes generating magnetic data at positions between the positions of the plurality of simulated sensors based on the respective magnetic fields at the positions of the plurality of simulated sensors.

In some implementations, taking the action based on the simulated magnetic data includes forwarding the simulated magnetic data of the organ of the subject for display on a graphical interface. The simulated magnetic data includes data corresponding to a respective magnetic field at a corresponding position of each of a plurality of simulated sensors near or around the current source. The simulated magnetic data can be configured to display via a graphical interface including at least one of: a magnetic field map at a time point, the magnetic field map including representative points of the plurality of simulated sensors at the corresponding positions near or around the current source, and an overlay or an average of the respective magnetic fields at the corresponding positions at the time point, a trace of the data corresponding to the respective magnetic fields over a period of time, or a trace of the current data corresponding to the current source over the period of time.

In some implementations, taking the action based on the simulated magnetic data includes: comparing the simulated magnetic data of the organ of the subject with measured magnetic data to obtain a comparison result; and re-generating the simulated magnetic data of the organ based on the electrical data of the organ of the subject and the comparison result.

In some implementations, the method includes: generating, by the one or more processors, simulated magnetic data of organs of subjects based on electrical data of the organs of the subjects; and storing, by the one or more processors, the generated simulated magnetic data of the organs of the subjects in a database.

In some implementations, the database includes corresponding magnetic data associated with respective organ-related diseases or organ-related biomarkers. Taking the action based on the simulated magnetic includes: comparing the simulated magnetic data of the organ of the subject with the corresponding magnetic data associated with respective organ-related diseases or organ-related biomarkers; and alerting a relevant party, e.g., a clinician licensed in the relevant jurisdiction, that the organ of the subject may be associated with a specific disease or a specific biomarker based on a result of the comparing.

Another aspect of the subject matter described in this present disclosure can be embodied in computer-implemented methods that include providing data of an organ of a subject as input to a machine learning system running on a computing system. The machine learning system includes at least one machine learning model that has been trained using organ-related simulated magnetic data of subjects. The actions further include in response to the input to the machine learning system, automatically generating a prediction result for the subject by the machine learning model based on the data of the organ of the subject. The actions further include taking an action based on the prediction result.

Other embodiments of these aspects include corresponding computer systems, apparatus, and computer programs recorded on one or more computer storage devices, each configured to perform the actions of the methods. A system of one or more computers to be configured to perform particular operations or actions means that the system has installed software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

In some implementations, the organ-related simulated magnetic data of the subjects are generated based on organ-related electrical data of the subjects by generating, for each piece of organ-related electric data, current data corresponding to a simulated electrical dipole source based on the piece of organ-related electrical data and generating corresponding organ-related simulated magnetic data based on the current data corresponding to the simulated electrical dipole source. In certain implementations, the simulated electrical dipole source is not fully reflective of, or is largely independent from, a physiological geometry of the organ.

In some implementations, the organ-related simulated magnetic data of the subjects includes corresponding magnetic data associated with respective organ-related diseases. The at least one machine learning model includes an organ-related disease classification model. The prediction result indicates, e.g., to a clinician licensed in the relevant jurisdiction, that the organ of the subject may have a specified disease.

In some implementations, the organ-related simulated magnetic data of the subjects includes corresponding magnetic data associated with respective organ-related biomarkers. The at least one machine learning model includes an organ-related biomarker identification model. The prediction result can indicate a biomarker identification result associated with the organ of the subject.

In some implementations, the data of the organ of the subject includes magnetic data of the organ measured with an unshielded magnetic sensing device. The at least one machine learning model includes a denoising model configured to denoise the magnetic data measured with the unshielded magnetic sensing device.

In some implementations, the actions further include training the denoising model by generating noisy magnetic data based on noise data and the organ-related simulated magnetic data of the subjects. The noise data is associated with a measurement environment where the magnetic data of the organ is measured with the unshielded magnetic sensing device. The actions further include training the denoising model by training the denoising model with the noisy magnetic data as the input of the denoising model and the organ-related simulated magnetic data of the subjects as output of the denoising model.

In some implementations, the denoising model includes a denoising autoencoder.

In some implementations, the organ-related simulated magnetic data of the subjects includes corresponding magnetic data associated with respective organ-related diseases or organ-related biomarkers. The at least one machine learning model includes at least one of an organ-related disease classification model or an organ-related biomarker identification model. The actions further include providing the denoised magnetic data outputted by the denoising model as input of the at least one of the organ-related disease classification model or the organ-related biomarker identification model. The actions further include automatically generating the prediction result by the at least one of the organ-related disease classification model or the organ-related biomarker identification model. The prediction result indicates at least one of a disease classification result associated with the organ of the subject or a biomarker identification result associated with the organ of the subject.

In some implementations, the actions further include receiving measured electrical data of the organ of the subject; generating current data corresponding to a simulated electrical dipole source based on the electrical data of the organ of the subject; and generating simulated magnetic data of the organ of the subject based on the current data corresponding to the simulated electrical dipole source. The data of the organ of the subject provided as the input to the machine learning system includes the generated simulated magnetic data of the organ of the subject.

In some implementations, the organ-related simulated magnetic data of subjects is stored in a repository accessible by the at least one machine learning model. The actions further include storing the generated simulated magnetic data of the organ of the subject and the prediction result in the repository and training the at least one machine learning model based on organ-related magnetic data in the repository including the generated simulated magnetic data of the organ of the subject.

In some implementations, the data of the organ of the subject includes ECG data of the organ of the subject. The at least one machine learning model includes an MCG transformation model that has been trained using a plurality of ECG datasets and a plurality of corresponding simulated MCG datasets based on the plurality of ECG datasets. The prediction result for the subject includes a corresponding simulated MCG dataset for the subject derived by the MCG transformation model based on the ECG data of the organ of the subject.

The details of one or more embodiments of the subject matter of the present disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example system for providing medical data, e.g., to a clinician.

FIG. 2A illustrates a flow chart of an example process for converting electrocardiography (ECG) data to simulated magnetocardiography (MCG) data.

FIG. 2B illustrates a flow chart of an example process for generating noisy simulated MCG data.

FIG. 3A illustrates an example 12-lead ECG measurement.

FIG. 3B illustrates an example of vectorcardiogram (VCG) data transformed from the 12-lead ECG measurement of FIG. 3A.

FIGS. 3C-3D illustrate example simulated MCG data determined based on the VCG data of FIG. 3B, where each trace corresponds to one of a plurality of simulated magnetic field sensors.

FIGS. 4A-4B illustrate example graphical interfaces showing simulated MCG data for healthy control patient (FIG. 4A) and patient with left ventricular hypertrophy (FIG. 4B).

FIG. 5 illustrates an example denoising machine learning (ML) model.

FIG. 6 illustrates an example feature extraction ML model.

FIG. 7 illustrates an example disease classification ML model.

FIG. 8 illustrates an example MCG transformation ML model.

FIG. 9A illustrates a flow chart of an example process of generating simulated magnetic data.

FIG. 9B illustrates a flow chart of an example process of using a ML system trained with simulated magnetic data.

FIG. 10 illustrates an example cloud server.

FIG. 11 illustrates an example computing system.

Like reference numbers and designations in the various drawings indicate like elements.

### DETAILED DESCRIPTION

Magnetocardiography (MCG) is a method for measuring the magnetic fields generated by the heart, which has been shown to provide highly sensitive and specific measurements for the diagnosis of various cardiovascular and metabolic conditions. Some implementations of the MCG techniques can use superconducting quantum interference devices (SQUID) or optically pumped magnetometers (OPMs) in shielded rooms to mitigate the impact of environmental noise. However, the large footprint and expense of the SQUID and the magnetic enclosures has severely limited its use, for example, in emergency rooms (ERs) where the majority of heart attacks (myocardial infarction) first present. Thus, smaller, unshielded, or mobile MCG devices operating at the point of care are needed to improve ER triaging of suspected myocardial infarction. One of the major challenges to the success of such a device is the dearth of data available to build the diagnostic algorithms. In addition, the signal-to-noise ratio of such devices is low due to noise interference. Thus, advanced analytic methods are required to discriminate diagnostically useful signals from the noise. Implementations of the present disclosure provide methods and systems that can leverage Artificial Intelligence (AI) and physics-based knowledge of the cardiovascular system to solve the above problems, thereby enabling deployment of a bedside unshielded MCG device.

Datasets large enough to create and evaluate generalizable AI algorithms on MCG data are desired. The MCG data used by some algorithms is on the order of tens or hundreds and is less than 500 participant-recordings in size in some instances. The dearth of data can be a problem because AI algorithms typically require large-scale data to achieve clinically acceptable metrics. Therefore, to develop generalizable AI algorithms that can perform well across numerous hospitals, ethnicities, states, and countries, significantly larger and more diverse datasets are desired. To close this data availability gap, some aspects of this disclosure provide methods and systems to convert 12-lead electrocardiography (ECG) recordings, which are plentiful and include clinical diagnostic labels, into clinically relevant simulated MCG recordings. The method of simulating MCG data can be used to bolster the diversity and size of datasets used to explore potential novel biomarkers, as well as train machine learning models to perform diagnostic tasks.

Some aspects of the present disclosure describe methods and systems for simulating magnetic data (e.g., MCG data) by leveraging methods of transforming abundant electrical data (e.g., ECG data, VCG data, and body surface potential mapping). Implementations of the present disclosure can provide one or more of the following technical advantages and/or benefits. The described techniques can take advantage of the expansive ECG-related datasets available from decades of researching, recording, and monitoring the electrical activity (e.g., through ECG) of both healthy and unhealthy individuals. These datasets can include data from various sources, including but not limited to open-source databases, academic databases, commercial databases, hospitals, healthcare providers, and other related institutions. Each dataset can have a large amount of data, e.g., each a single hospital could, over the course of 10 years, gather more than 5 million ECG recordings. These methods are flexible in their implementation and can simulate and record data from a wide variety of theoretical configurations of sensor positions near or around an approximated cardiac dipole source. In some implementations, when transforming ECG into VCG, the 2D to 3D transformation may provide benefits that can be harvested via AI/ML methods. Specifically, although the information space would seem to remain unchanged, a 3D representation may lend itself to well-honed AI image-processing methodologies that can deconvolute or otherwise distribute portions of the relevant signal across a spatial-temporal map, which then allows for superior AI predictions.

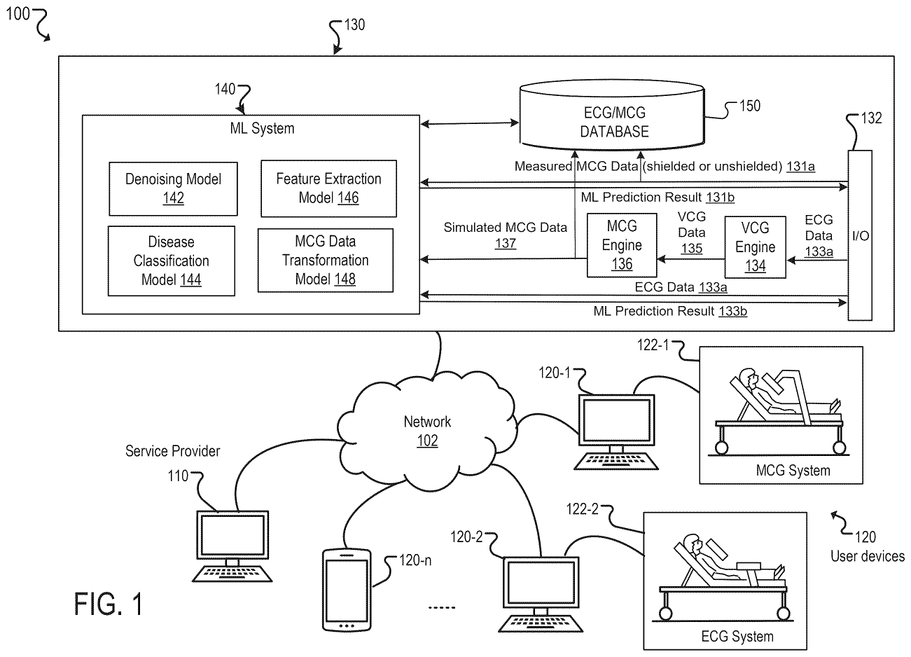

FIG. 1 illustrates an example system 100 for providing medical data, e.g., to a clinician. The system 100 can include a network 102, a service provider system 110, one or more user devices 120, and a cloud computing platform 130. The network 102 can include a large computer network, such as a local area network (LAN), a wide area network (WAN), the Internet, a cellular network, or a combination thereof, connecting any number of mobile computing devices, fixed computing devices, and server systems. The user devices 120 can be associated with one or more corresponding users. In some implementations, the user devices 120 can include user computers 120-1, 120-2, . . . , and a mobile device 120-n. The service provider system 110 can be associated with a corresponding service company or business entity. In some implementations, the service provider system 110 can communicate with users via any type of devices, systems, or servers, e.g., a desktop computer, a mobile device, a smart mobile phone, a tablet computing device, a notebook, or a portable communication device.

In some implementations, the cloud computing platform 130 can be hosted by the service provider system 110. In some implementations, the system 100 can deploy a Software as a Service (SaaS) model. SaaS is a software distribution model in which a cloud provider (e.g., the cloud computing platform 130) hosts applications and makes them available to end users (e.g., the user devices 120) over the network 102. In this model, an independent software vendor (ISV) (e.g., the service provider system 110) may contract a third-party cloud provider (e.g., Amazon web services (AWS), Microsoft Azure, Google cloud platform (GCP)) to host an application. Users can access the application through a web browser without needing to install or maintain it locally on their own computers. Examples of SaaS can include productivity tools like Google Workspace, customer relationship management (CRM) systems like Salesforce, and project management platforms like Asana. With larger companies, such as Microsoft, the cloud provider might also be a software vendor.

The system 100 can further include one or more medical devices or systems (e.g., systems 122-1 or 122-2) that are configured to measure electrical/magnetic data of an organ of a subject (e.g., a human patient). Each of the medical systems can be coupled to one of the user devices 120. The medical systems can measure the organ's electrical activity by using electrodes placed on the skin of the subject or by recording a magnetic field that the organ produces. For example, as shown in FIG. 1, the user device 120-1 can be coupled to a magnetocardiography (MCG) system 122-1. The MCG system 122-1 can include one or more magnetometers (e.g., optically pumped magnetometers (OPMs)) that are configured to measure a magnetic signal from the heart of the subject. In some implementations, the magnetic signal is a magnetic flux density. In some instances, the MCG system 122-1 (e.g., SQUID) can be operating in a magnetically shielded room to mitigate the impact of environmental noise. In some other instances, the MCG system 122-1 can be operating in a magnetically unshielded environment. In some implementations, as shown in FIG. 1, the user device 120-2 can be coupled to an electrocardiography (ECG) system 122-2. The ECG system 122-2 can produce an electrocardiogram, a recording of the heart's electrical activity through repeated cardiac cycles, using electrodes placed on the subject's skin. In some other implementations (not shown in FIG. 1), the user device 120 can be coupled to a magnetoencephalography (MEG) system that measures magnetic fields produced by electrical activities of the subject's brain.

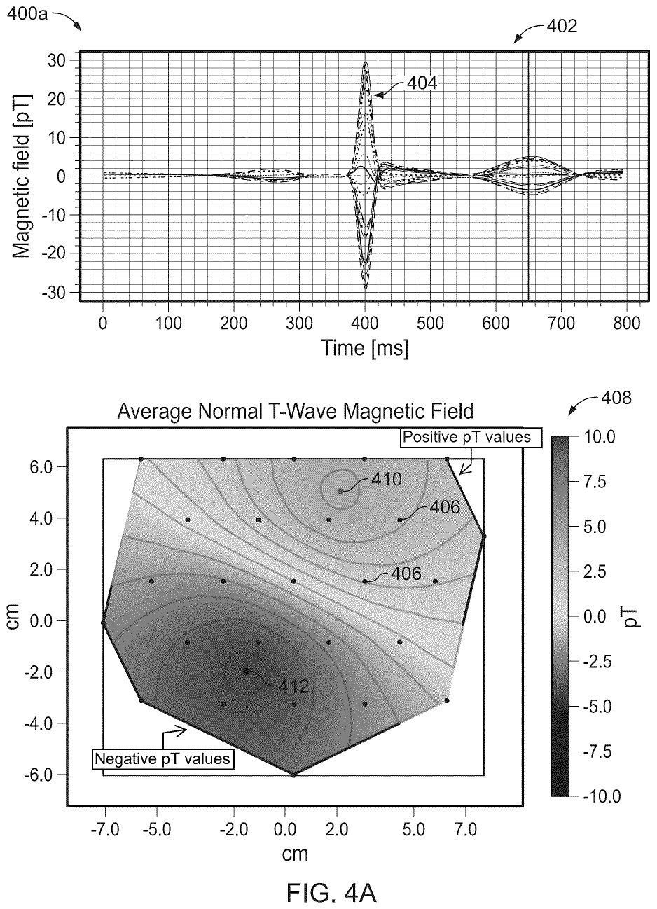

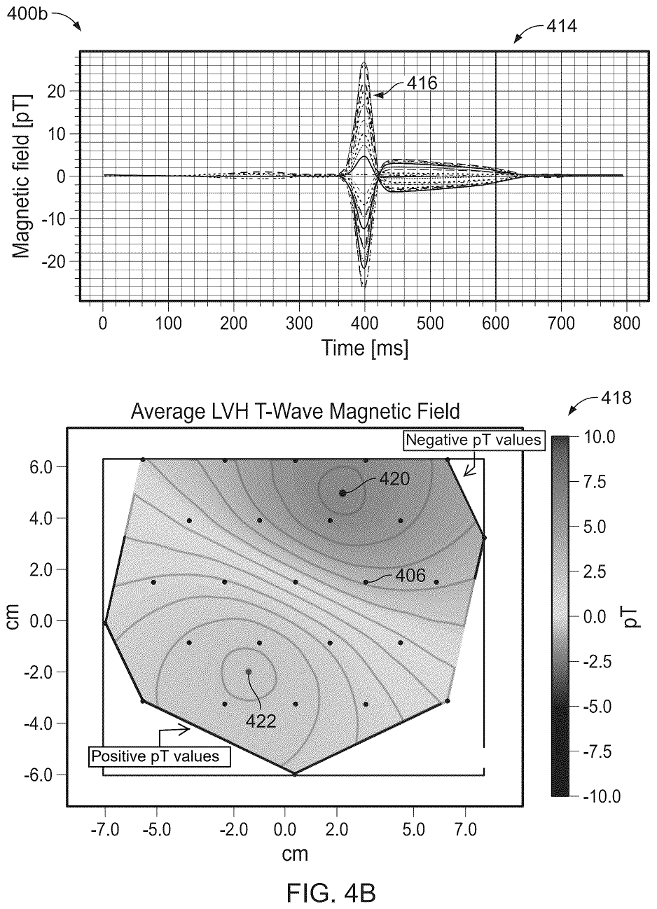

In some implementations, measured data generated by the medical systems (e.g., the MCG system 122-1 or the ECG system 122-2) coupled to the user devices 120 are transmitted (e.g., through the user device 120 and the network 102) to the cloud computing platform 130 for further processing. The cloud computing platform 130 can receive input data from the network 102 and generate results (e.g., results 131b or 133b as shown in FIG. 1) based on at least the input data. In some implementations, the input data of the cloud computing platform 130 can include the measured data generated by the medical systems (e.g., the MCG system 122-1 or the ECG system 122-2). In some other implementations, the input data can be obtained from an existing database or can be generated by an exterior medical system that is not included in the system 100. In some implementations, the input data (e.g., the measured data) and/or the results generated by the cloud computing platform 130 can be displayed on the service provider system 110 or any of the user devices 120. For example, the user device 120 can be caused to present a graphical interface corresponding to the input data and/or the results generated by the cloud computing platform 130. Examples of the graphical interface are described below in further detail with reference to FIGS. 4A-4B.

The cloud computing platform 130 can include one or more computing devices and one or more machine-readable repositories, or databases. In some implementations, the cloud computing platform 130 can include one or more server computers in a local or distributed network each having one or more processing cores. The cloud computing platform 130 can be implemented in a parallel processing or peer-to-peer infrastructure or on a single device with one or more processors.

The cloud computing platform 130 can include an input/output (I/O) interface 132, a vectorcardiogram (VCG) engine 134, an MCG engine 136, a machine learning (ML) system 140, and an ECG/MCG database 150. The I/O interface 132 can be configured to receive the input data of the cloud computing platform 130 from the network 102 and to transmit the results generated by the cloud computing platform 130 to the network 102. The ML system 140 can be configured to perform ML processing based on the input data of the cloud computing platform 130. The ML system 140 can include one or more ML models (e.g., a denoising ML model 142, a disease classification ML model 144, a feature extraction ML model 146, or an MCG transformation ML model 148 as shown in FIG. 1). In some implementations, the ML system 140 can train the ML models based on the input data of the cloud computing platform 130. The ECG/MCG database 150 can be configured to store ECG data and/or MCG data, which can include the input data and/or data generated based on the input data.

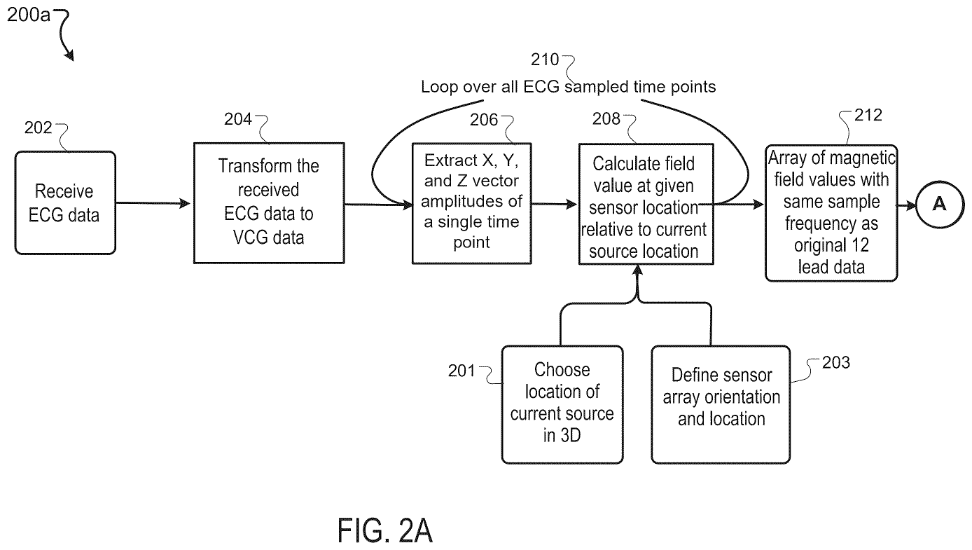

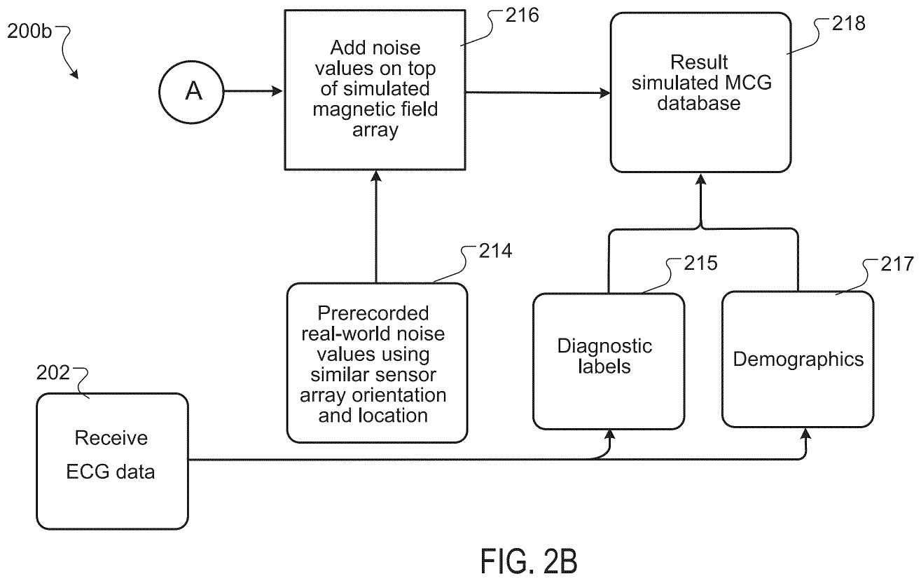

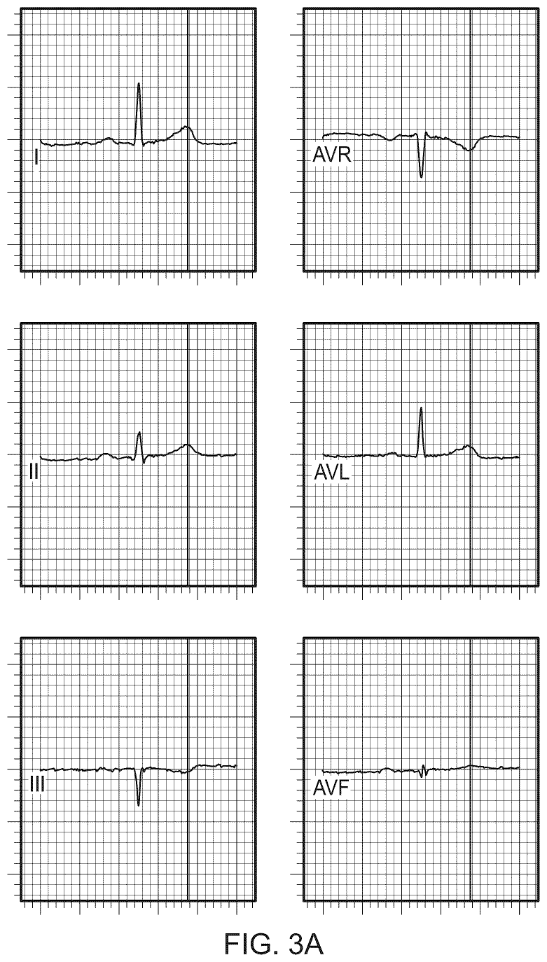

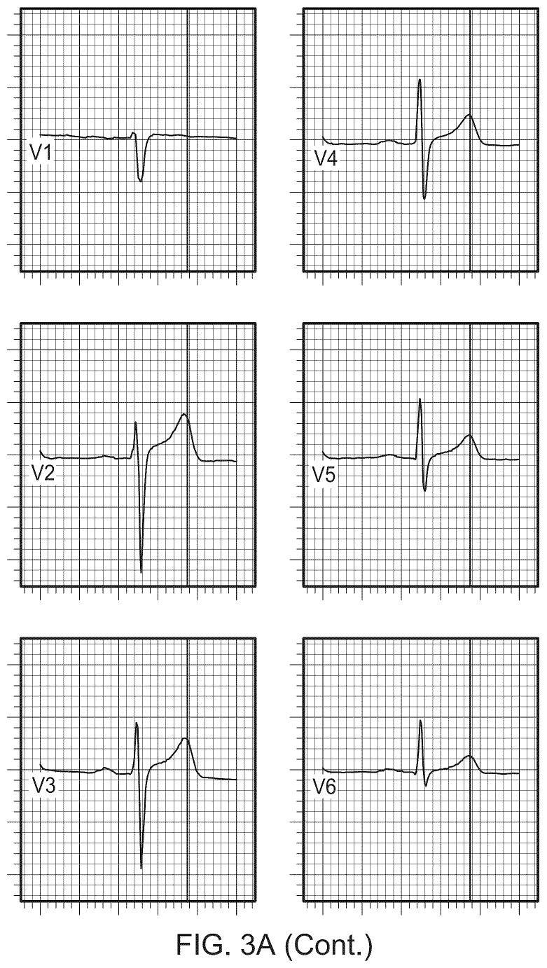

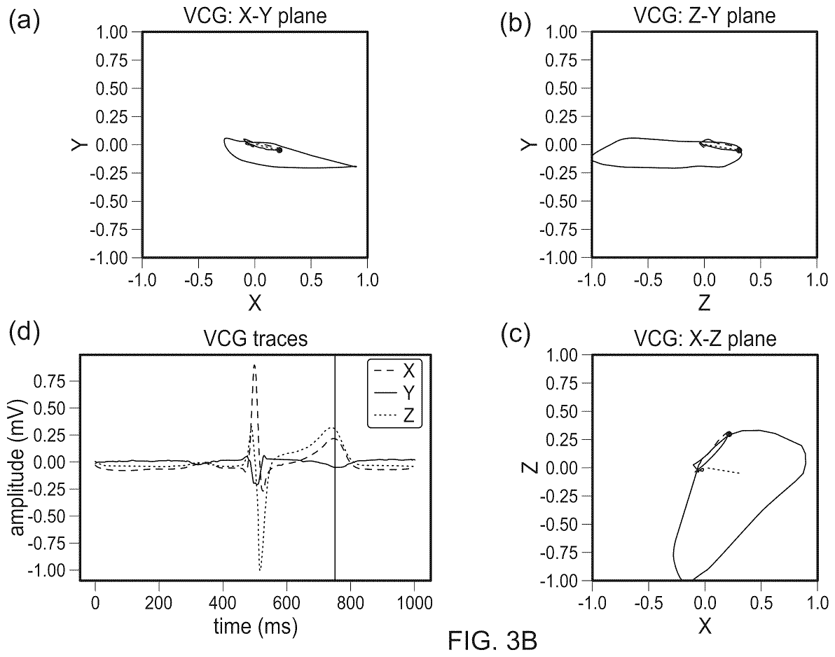

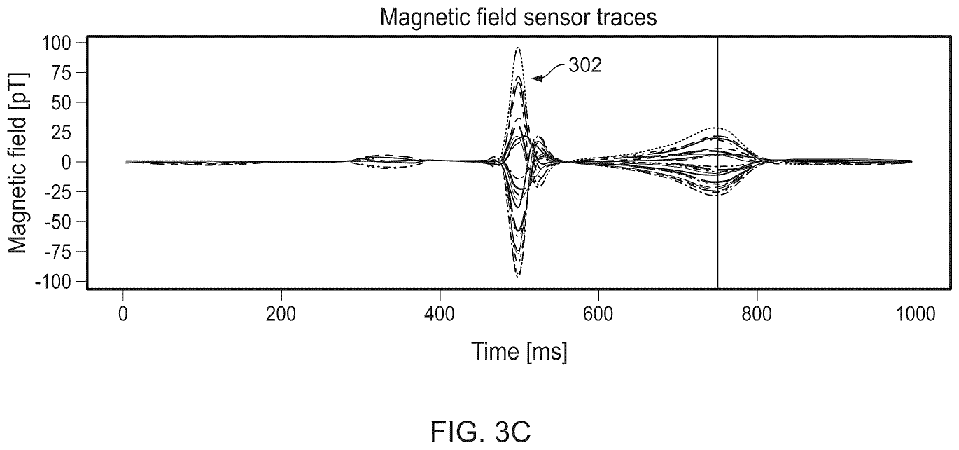

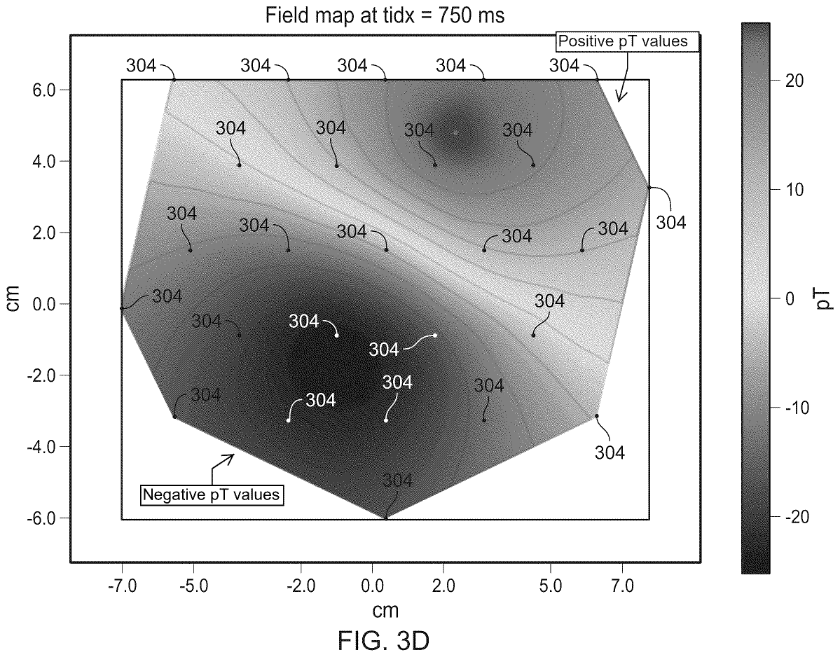

In some implementations, the input data can include ECG data 133a of an organ of a subject. The ML system 140 can generate a ML prediction result 133b based on the ECG data 133a. In some instances, the ECG data 133a can be generated by the ECG system 122-2 of the system 100. In some other instances, the ECG data 133a can be obtained from an existing ECG database (e.g., an open-source or commercial database that includes ECG records). The cloud computing platform 130 can generate simulated MCG data 137 based on the ECG data 133a and generate a training database by accumulating a large amount of the simulated MCG data 137. In one example, the VCG engine 134 can generate VCG data 135 based on the ECG data 133a, and the MCG engine 136 can generate the simulated MCG data 137 based on the VCG data 135. Generation of the simulated MCG data 137 using the VCG engine 134 and the MCG engine 136 is described below in further detail with reference to FIGS. 2A-3D.

The simulated MCG data 137 can be stored in the ECG/MCG database 150 and can be used to train the ML models of the ML system 140. In one example, once a sufficiently large dataset (e.g., including the ECG data 133a and the simulated MCG data 137 generated based on the ECG data 133a) is generated using the VCG engine 134 and the MCG engine 136 described above, the MCG transformation ML model 148 can be trained using the generated dataset to directly transform ECG data to MCG data (e.g., for a given sensor configuration). As such, when the cloud computing platform 130 receives new ECG data 133a, the MCG transformation ML model 148 can be applied to the new ECG data 133a to generate corresponding simulated MCG data 137. In some implementations, applying the MCG transformation ML model 148 may utilize less computational resources than the VCG engine 134 and the MCG engine 136. In some implementations, the MCG transformation ML model 148 can use any suitable hyperparameters, such as parameters for simulating a linear or a non-linear regression network. In some instances, the MCG transformation ML model 148 uses more hyperparameter when simulating a non-linear regression network than it does when simulating a linear regression network. In some implementations, the MCG engine 136 can perform forward modeling using current-element integration, magnetic-dipole modeling, or a combination of both based on the simulation requirements, as described in further detail below with reference to FIG. 2A.

In some implementations, the cloud computing platform 130 can receive both the ECG data 133a of the organ of the subject and the measured MCG data 131a. For example, the ECG data 133a and the measured MCG data 131a can be received at the same time. The measured MCG data 131a can be MCG data of the organ of the same subject. In some implementations, the measured MCG data 131a can be representative data of the same organ but from different subject(s). The cloud computing platform 130 can compare the simulated MCG data 137 generated based on the ECG data 133a with the measured MCG data 131a to obtain a comparison result. The comparison result can be used for validation purposes, for example, to evaluate or improve the accuracy of the conversion from the ECG data 133a to the simulated MCG data 137. In some implementations, the comparison can be based on a biomarker of the simulated MCG data 137 and the measured MCG data 131a. For example, the cloud computing platform 130 can evaluate whether the simulated MCG data 137 and the measured MCG data 131a have similar T-wave field max angles. Parameters used by the VCG engine 134 and the MCG engine 136 or the MCG transformation ML model 148 can be adjusted based on the comparison result. The parameters can include, for example, a transformation matrix for the ECG to VCG conversion, weights applied to the transformation of certain sections of the ECG data (e.g., R-peak, T-wave), various “earth field” values imposed on the simulation, parameters associated with simulating multiple current dipoles, or any suitable combination thereof. In some implementations, the adjusted VCG engine 134, MCG engine 136, or MCG transformation ML model 148 can be used to re-generate the simulated MCG data 137 based on the ECG data 133a and can have improved performance.

In some implementations, the simulated MCG data 137 can be used to train or refine one or more of the denoising model 142, the disease classification model 144, and the feature extraction model 146. In some implementations, the disease classification model 144 and the feature extraction model 146 can be applied to the simulated MCG data 137. For example, the disease classification model 144 can be used to compare the simulated MCG data 137 of the organ of the subject with corresponding MCG data in the ECG/MCG database 150 that is associated with a respective organ-related disease. In addition, the disease classification model 144 can be configured to determine whether the organ of the subject is associated with a specific organ-related disease based on a comparison result. In another example, the feature extraction model 146 can be used to compare the simulated MCG data 137 of the organ of the subject with corresponding MCG data in the ECG/MCG database 150 that is associated with a respective organ-related biomarker. In addition, the feature extraction model 146 can be configured to determine whether the organ of the subject is associated with a specific organ-related biomarker based on a comparison result. In some other implementations, the feature extraction model 146 can be used to determine potential biomarkers based on the simulated MCG data 137. In some implementations, the ML prediction result 133b can include the simulated MCG data 137, the specific organ-related disease determined by the disease classification model 144 based on the simulated MCG data 137, the specific organ-related biomarker or the potential biomarker determined by the feature extraction model 146 based on the simulated MCG data 137, or any combination thereof.

In some implementations, the input data can include measured MCG data 131a of an organ of a subject. For example, the measured MCG data 131a can be unshielded MCG data generated by the MCG system 122-1 (e.g., an unshielded MCG system) in a magnetically unshielded environment. In another example, the measured MCG data 131a can be shielded MCG data generated by the MCG system 122-1 (e.g., SQUID) in a magnetically shielded environment. The measured MCG data 131a can be stored in the ECG/MCG database 150 and can be used for training or refining the ML models of the ML system 140. In addition, the ML system 140 can apply one or more of the ML models (e.g., the denoising model 142, the disease classification model 144, or the feature extraction model 146) to the measured MCG data 131a. For example, when the measured MCG data 131a is the unshielded MCG data, the denoising model 142 can be used to generate denoised MCG data by removing magnetic noise signals in the measured MCG data 131a. In some implementations, the denoised MCG data can also be stored in the ECG/MCG database 150.

When the measured MCG data 131a is the shielded MCG data, the disease classification model 144 and the feature extraction model 146 can be applied to the measured MCG data 131a. In some implementations, the disease classification model 144 and the feature extraction model 146 can also be applied to the denoised MCG data generated by the denoising model 142. The disease classification model 144 can be used to compare the measured MCG data 131a of the organ of the subject with corresponding MCG data in the ECG/MCG database 150 that is associated with a respective organ-related disease. In addition, the disease classification model 144 can be configured to provide data to a clinician to determine whether the organ of the subject is associated with a specific organ-related disease based on a comparison result. In another example, the feature extraction model 146 can be used to compare the measured MCG data 131a of the organ of the subject with corresponding MCG data in the ECG/MCG database 150 that is associated with a respective organ-related biomarker. In addition, the feature extraction model 146 can be configured to determine whether the organ of the subject is associated with a specific organ-related biomarker based on a comparison result. In some implementations, the ML prediction result 131b can include the specific organ-related disease determined by the disease classification model 144 based on the measured MCG data 131a, the specific organ-related biomarker determined by the feature extraction model 146 based on the measured MCG data 131a, or any combination thereof.

In some implementations, the input data includes simulated magnetic data of an organ of a subject. The simulated magnetic data of the organ can be simulated MCG data for a heart. For example, the computing device 120-1 can include a VCG engine (e.g., the VCG engine 134) and an MCG engine (e.g., the MCG engine 136). After receiving measured ECG data from an ECG system 122-1, the computing device 120-1 can generate the simulated MCG data based on the measured ECG data and transmit the simulated MCG data to the cloud computing system 130. The cloud computing system 130 can store the simulated MCG data in the ECG/MCG database 150. The cloud computing system 130 can also use one or more ML models in the ML system 140 to generate a prediction result 131b, as noted above.

FIG. 2A illustrates a flow chart of an example process 200a for converting ECG data to simulated MCG data. In some implementations, the process 200 can be performed by the cloud computing platform 130 (e.g., the VCG engine 134 and the MCG engine 136) of FIG. 1. As shown in FIG. 2A, at operation 202, a VCG engine (e.g., the VCG engine 134 of FIG. 1) receives or obtains ECG data (e.g., ECG data 133a of FIG. 1) of a heart of a subject. The ECG data can be generated by the ECG system 122-2 of FIG. 1 using leads or electrodes attached to the body of the subject. FIG. 3A shows an example 12-lead ECG measurement. The twelve subfigures of FIG. 3A are different traces included in the 12-lead ECG measurement. As shown in FIG. 3A, these traces are labeled with their respective lead names, such as leads I, II, III, AVF, V3, etc.

At operation 204, the VCG engine transforms the received ECG data to VCG data (e.g., the VCG data 135 of FIG. 1). In some implementations, the transformation can be based on at least one of Kors Regression, Kors quasi-orthogonal, inverse Dower, or a linear regression-based transformation. The VCG data can be a representation of an average direction of currents flowing in the heart at a given point in time. The VCG operates under the assumption that we are reducing/averaging all the current sources in the heart down to one, single current source with a magnitude and direction. In one example, the VCG data can be a three-dimensional (3D) vector (also referred to as a VCG vector) and can be a representation of the ECG data in 3D coordinates (e.g., right-left axis (X), head-to-feet axis (Y) and front-back (anteroposterior) axis (Z)) of the human body. FIG. 3B shows an example of the VCG data transformed from the 12-lead ECG measurement of FIG. 3A using Kors Regression. FIG. 3B(d) shows the VCG vector in each of the three coordinates during a time window between 1 ms and 1000 ms. At any point in time, the VCG vector can be represented as an arrow pointing in a direction of the VCG vector in X-Y-Z space, and a length of the arrow represents the magnitude of the VCG vector. A series of loops for each heartbeat can be obtained by making a trace following the tip of this VCG vector. The loops can describe the direction and magnitude of the electrical current of the heart over time. FIGS. 3B(a)-3B(c) show some loops of such a trace generated based on the VCG vector within the time window as shown in FIG. 3B(d). FIG. 3B(a) illustrates a projection of the trace onto the X-Y plane, FIG. 3B(b) illustrates a projection of the trace onto the Z-Y plane, and FIG. 3B(c) illustrates a projection of the trace onto the X-Z plane.

An MCG engine (e.g., the MCG engine 136 of FIG. 1) can generate current data corresponding to a current source based on the VCG data (e.g., as shown in FIG. 3B) where the current source is approximated as a straight wire carrying a current. The length of the VCG vector can represent the magnitude of the current and the direction of the VCG vector represent the direction of the current source at a particular moment in time.

The MCG engine can generate the simulated MCG data (e.g., the simulated MCG data 137 of FIG. 1) based on the current data corresponding to the current source. In this example, the organ is the heart, and the current data includes vector data that represents electrical current data of the heart. The MCG engine 136 can generate a set of vector data for each of a plurality of heart beats of the subject. In some implementations, the current source is derived by approximating an electrical current of the organ of the subject as a set of current dipoles. For example, in certain implementations, an electrical activity of the organ at a single time point is represented by a corresponding single current dipole of the set of current dipoles. In some implementations, the current source is a single current dipole. Alternatively, the current source can be a series of current dipoles. The simulated MCG data of the organ of the subject can be associated with a synthetic result of magnetic fields from the set of current dipoles of the current source.

The described techniques can simplify the electrical activity of the entire heart into the current source. The current source does not fully represent, e.g., can be largely independent from, a physiological geometry of the organ of the subject. That is, even though the received ECG data may be associated with different subjects or patients, the MCG engine can treat the current data as if it is gathered from the same subject, and thus can generate representative data of a magnetic field of the organ of the subject based on the current data. In some implementations, the simulated MCG data can include the representative data of the magnetic field. The MCG engine can generate the representative data of the magnetic field of the organ of the subject based on the current data by physics-based forward modeling.

Returning to FIG. 2A, operations 201, 203, 206, 208, 210, and 212 can be performed by the MCG engine 136 to transform the VCG data to the simulated MCG data (e.g., the simulated MCG data 137 of FIG. 1). At the operation 206, corresponding vector amplitudes of the VCG data along axes (e.g., the X, Y, and Z coordinates as shown in FIG. 3B) of a coordinate system of a vector representing the electric data at a single time point is extracted. At the operation 208, the MCG engine 136 can convert the VCG data to the simulated MCG data at a specified point in time. The MCG engine 136 can calculate a magnetic field value at a given sensor location relative to the current source in the coordinate system. In the example of FIG. 2A, the current data is a single current dipole. The conversion can be calculated using a Biot-Savart equation by integrating the current data along a path C of the current source according to the Biot-Savart equation expressed as:

$$B ( r ) = \frac{{μ}_{0}}{4 π} {\int }_{C} \frac{Idl \times {r}^{′}}{{\vert {r}^{′} \vert}^{3}}$$

where μ₀ is permeability of free space, r represents a radius away from the current source, B(r) is a magnetic field generated by a given current at the radius r away from the current source, dl represents an infinitesimal section of the current source, I is a current magnitude of that section of the current source, and C is the path of the current source. In some implementations, the path of the current source over a short, e.g., millisecond, timeframe, can be considered as a straight line, dl, and I can be determined by the VCG data (e.g., a VCG vector at a given time). For example, dl can be the same as the direction of the VCG vector at a specified time, and I can be the magnitude of the VCG vector, e.g., adjusted by a scaling factor.

Given a location of the current source and locations of magnetic measurement sensors (e.g., a simulated or virtual sensor array) near or around the current source, the simulated MCG data at that time point can be calculated based on the above equation. Alternatively, in some implementations (e.g., as shown in the function of Table 1), the simulated MCG data at that time point can be calculated using the coefficient

$$\frac{{μ}_{0}}{4 π}$$

and the function

$$\frac{Idl \times {r}^{′}}{{\vert {r}^{′} \vert}^{3}}$$

inside the Biot-Savart equation without evaluating the integral. In some implementations, as shown in operation 201, the location of the current source in the 3-D space can be chosen. For example, the current source can be placed at a location of the heart (e.g., the center of the heart). In some implementations, as shown in operation 203, the orientation and location of the simulated sensor array can also be chosen. As shown in operation 210, the MCG engine 136 can loop the operations 206 and 208 over some or all sampled time points (e.g., sampled time points during the ECG measurement) and generate the simulated MCG data over the sampled time points. The simulated MCG data can include a group of magnetic field values or traces. Each trace in the group of magnetic field traces can be associated with one sensor of the simulated sensor array. An example function that calculates a magnetic field vector at a given point resulting from a current dipole (also can be considered as a current source) is provided in Table 1 below. As shown in the function, parameter r represents a location of a simulated sensor, parameter rp represents a location of the current source, parameter idl represents a magnitude and a direction of a current flowing in the current source. As described above, the parameter idl can be determined based on the VCG data at the given point. A return value Bdip represents the magnetic field vector measured by the simulated sensor, which is determined by applying the coefficient

$$\frac{{μ}_{0}}{4 π}$$

of the Biot-Savart equation and the function

$$\frac{Idl \times {r}^{′}}{{\vert {r}^{′} \vert}^{3}}$$

inside the Biot-Savart equation to the parameters r, rp, and idl without evaluating the integral. While in this example, the function is written using python, it is understood that any suitable programming language can be used in practice.

```
TABLE 1
def B_current_dipole(r: np.array, rp: np.array, idl: np.array):
  “““
  Returns the vector magnetic field at a position r from a current dipole
  idl located at rp.
  Parameters
  ----------
  r : np.array shape (3.)
    Position where magnetic field is being observed. [r] = meters
  rp : np.array shape (3.)
    Location of magnetic dipole idl. [rp] = meters
  idl : np.array shape (3.)
    current dipole located at rp. [idl] = A meter
  Returns
  -------
  output: np.array shape (3.)
    Magnetic field measured at r from dipole idl located at rp. Units are nT
  ”””
  if r.ndim > 1:
    raise ValueError(
      “Input r vector has dimensions higher than 1. Function expects : r : np.array
shape (3.)”
    )
  if rp.ndim > 1:
    raise ValueError(
      “Input rp vector has dimensions higher than 1. Function expects : rp : np.array
shape (3.)”
    )
  if idl.ndim > 1:
    raise ValueError(
      “Input idl vector has dimensions higher than 1. Function expects : idl : np.array
shape (3.)”
    )
  mu0 = 4 * np.pi * 1e−7
  R = r − rp
  Rmag = np.linalg.norm(R)
  Bdip = mu0 / (4 * np.pi) * (np.cross(idl, R)) / (Rmag**3) * 1e9
  return Bdip
```

In some implementations, the forward modeling can alternatively or additionally be based on one or more magnetic-dipole sources. A magnetic dipole source can be represented by a magnetic-moment vector m, and the magnetic field B(r) generated by a given current at a given position r away from the current sources can be expressed as:

$$β ( r ) = ∇ \times A = \frac{{μ}_{0}}{4 π} [ \frac{3 r ( m \cdot r )}{{r}^{5}} - m / {r}^{3} ]$$

where A is the magnetic vector potential, μ₀ is permeability of free space, and r represents a radius away from the current source. The system can use this formulation to better approximate the magnetic field generalized by localized current loops, the effective current distributions within the myocardium, or both. In some examples, the simulation can combine magnetic dipole and current-element integrations to capture both distributed and localized cardiac sources. The resulting simulated magnetic field values can then be sampled at positions corresponding to an array of MCG sensors to generate synthetic MCG data.

At operation 212, the group of magnetic field traces can be further processed so that these traces have the same sample frequency as the ECG measurement.

FIGS. 3C-3D illustrates an example of simulated MCG data determined based on the VCG data of FIG. 3B. As shown in FIG. 3C, the simulated MCG data includes a group of magnetic field traces 302. Each magnetic field trace in the group of magnetic field traces 302 can be considered as magnetic field values measured by one of the simulated sensors 304 (as shown in FIG. 3D) over the time window between 0 ms and 1000 ms. The simulated sensors 304 can form an array, and FIG. 3D illustrates that a location of each simulated sensor 304 relative to the location of the current source. FIG. 3D also illustrates a magnetic field map (MFM) of the current source at a time point 750 ms, which can be determined using interpolation methods based on the group of magnetic field traces 302 and the locations of the simulated sensors 304. For example, the MFM of the current source can be determined using a cubic interpolation from a python package for 2D interpolation. In this example, the simulated sensors 304 are located in a 2D plane (e.g., the X-Y plane at a fixed position along the Z axis).

FIG. 2B illustrates a flow chart of an example process 200b for generating noisy simulated MCG data. At operation 216, noise signals can be added on top of the simulated MCG data (e.g., output of the operation 212 of FIG. 2A). As shown in operation 214, the noise signals can be prerecorded real-world noise values based on a sensor array whose orientation and location are similar to the simulated sensors 304 described above. For example, the noise signals can be a magnetic measurement of an ambient or background environment when the subject is absent. At operation 218, the noisy simulated MCG data can be generated. In some implementations, as shown in operations 215 and 217, information such as diagnostic labels and demographics that are included in the received ECG data (e.g., at operation 202) also can be added to the noisy simulated MCG data. In this way, the noisy simulated MCG data and the diagnostic labels and demographics that the noisy simulated MCG data is associated with can also be used for training or refining the ML models of the ML system 140.

FIGS. 4A-4B illustrate an example of the graphical interfaces that can be displayed on the service provider system 110 or any of the user devices 120. In some implementations, the system 100 can cause the service provider system 110 or any of the user devices 120 to present a graphical interface corresponding to simulated magnetic data (e.g., the simulated MCG data 137 of FIG. 1) of the organ of the subject. The simulated magnetic data can include data corresponding to a respective magnetic field at a corresponding position of each of a plurality of simulated sensors (e.g., the simulated sensors 304 of FIG. 3D) near or around the current source. The graphical interface can include at least one of: a magnetic field map (MFM) at a time point, a trace of the data corresponding to the respective magnetic fields over a period of time, or a trace of the current data corresponding to the current source over the period of time. In some implementations, the magnetic field map (MFM) includes representative points of the plurality of simulated sensors at the corresponding positions near or around the current source, and an overlay or an average of the respective magnetic fields at the corresponding positions at the time point.

FIG. 4A illustrates an example graphical interface 400a, which shows both simulated MCG data 402 and an MFM 408. The simulated MCG data 402 are generated based on ECG data (over a time window between 0 ms and 800 ms) of a healthy patient. The simulated MCG data 402 includes a group of magnetic field traces 404. Each magnetic field trace in the group of magnetic field traces 404 can be considered as magnetic field values measured by one of the simulated sensors 406 (as shown in FIG. 4A) over the time window between 0 ms and 800 ms. The simulated sensors 406 can form an array, and FIG. 4A illustrates the locations of the simulated sensors 406 in relative to a location of a current source for generating the simulated MCG data 402. FIG. 4A also illustrates the MFM 408 of the current source at time point 650 ms. As shown in FIG. 4A, the MFM 408 has a positive magnetic pole 410 and a negative magnetic pole 412. In some implementations, the graphical interface 400a can display a process of the MFM 408 varying during the time window. In some implementations, the graphical interface 400a can interact with a user or a viewer and allow the user to select a time point in the time window. The graphical interface 400a can then display the MFM 408 at the time point selected by the user.

FIG. 4B illustrates another example graphical interface 400b, which is similar to the graphical interface 400a. The graphical interface 400b shows both simulated MCG data 414 and a MFM 418. The simulated MCG data 414 are generated based on ECG data (over a time window between 0 ms and 800 ms) of a patient with left ventricular hypertrophy (LVH). The simulated MCG data 414 includes a group of magnetic field traces 416. Each magnetic field trace in the group of magnetic field traces 416 can be considered as magnetic field values measured by one of the simulated sensors 406 (same as in FIG. 4A) over the time window between 0 ms and 800 ms. FIG. 4B also illustrates the MFM 418 of the current source at time point 600 ms. As shown in FIG. 4B, the MFM 418 has a positive magnetic pole 422 and a negative magnetic pole 420.

The graphical interfaces 400a and 400b can provide information that allows the user to associate the simulated MCG data and the MFM with a potential disease. For example, in FIG. 4A, the south magnetic pole 410 is located on the top section of the MFM 408, and the north magnetic pole 412 is located on the bottom section of the MFM 408. On the contrary, as shown in FIG. 4B, the MFM 418 has the positive magnetic pole 422 on its bottom section and the negative magnetic pole 420 on its top section. In other words, the MFM 418 of FIG. 4B has inverted magnetic poles or polarity compared to the MFM 408 of FIG. 4A. Such a comparison result may suggest that the patient of the MFM 418 has LVH since LVH patients can have inverted T-waves.

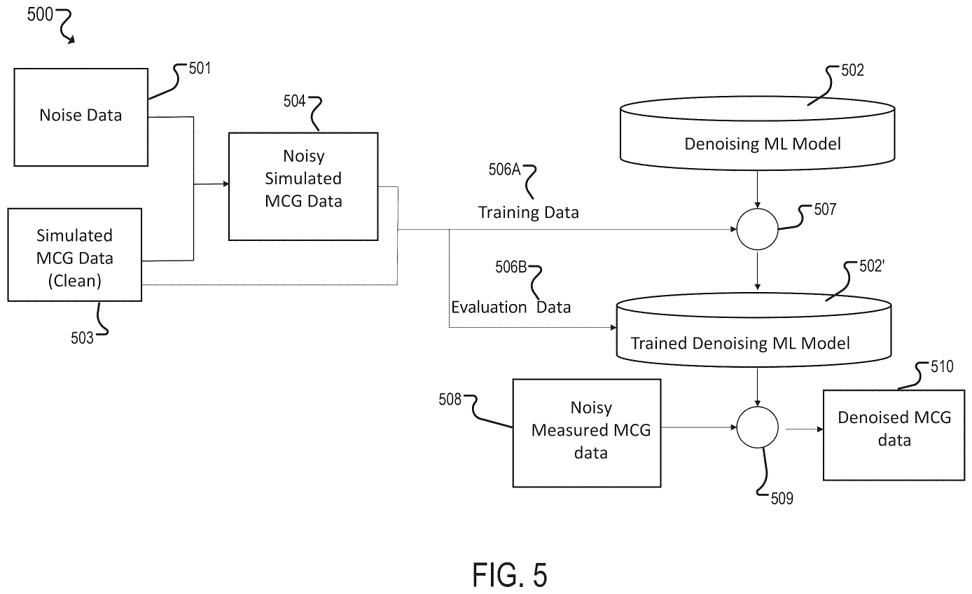

FIG. 5 is a schematic diagram illustrating an example denoising process 500 performed by a ML system. The ML system can be an example of the ML system 140 of FIG. 1. The denoising process 500 can be implemented by a computing system (e.g., the cloud computing platform 130 of FIG. 1) that includes one or more computing devices and one or more machine-readable repositories or databases that are in communication with each other.

The ML system can include one or more denoising ML models 502. The denoising ML models 502 can be examples of the denoising ML model 142 of FIG. 1. For example, the denoising ML models 502 can include a denoising autoencoder. The denoising autoencoder can be a U-net, which is a convolutional neural network developed for biomedical image processing. In some implementations, the denoising autoencoder can be a diffusion-based autoencoder, which is a type of generative model that combines elements of diffusion processes with autoencoder architectures. In some implementations, simulated MCG data 503 and noisy simulated MCG data 504 are prepared for training and/or evaluating the denoising ML models 502. In some implementations, the computing system can store the simulated MCG data 503 and the noisy simulated MCG data 504 in a repository (e.g., the ECG/MCG database 150 of FIG. 1) accessible by the one or more denoising ML models 502. The simulated MCG data 503 and the noisy simulated MCG data 504 can be divided into training data 506A and evaluation data 506B. The simulated MCG data 503 can be clean simulated MCG data associated with an organ of a subject. The clean simulated MCG data may include little or no noise data. For example, the simulated MCG data 503 can be the simulated MCG data 137 of FIG. 1 that is generated based on the ECG data 133a. The noisy simulated MCG data 504 (also referred to as overlaid simulated MCG data) can be generated by adding or overlaying noise data 501 to the simulated MCG data 503. In some implementations, the noise data 501 can be associated with an environmental noise signal caused by a natural ambient environment. In this case, the noise data 501 can be generated by measuring or recording background noises in a target environment when the subject is absent. In some implementations, the noise data 501 can be associated with a motional noise signal caused by motions of magnetometers of an MCG system (e.g., the MCG system 122-1 of FIG. 1) when the MCG system was taking magnetic measurements. In this case, a motional measurement of the magnetometers can be obtained. For example, the motional measurement can include acceleration and rotational information of each of the magnetometers. The noise data 501 can be generated based on the magnetic measurements of the MCG system and the motional measurement. In some implementations, the noise data 501 can be associated with a malfunction or a failure of one of the magnetometers. In this case, the noise data 501 can be generated by determining an elevated noise floor in the magnetic measurements. In some implementations, the noise data 501 can be generated by any suitable combination of the above-mentioned methods.

At a processing step 507, the computing system can train one of the denoising ML models 502 with respect to the training data 506A and return a trained denoising ML model 502′. In some implementations, after training the denoising ML model 502 with the training data 506A, the trained denoising ML model 502′ is evaluated with the evaluation data 506B. The evaluation data 506B can provide independent measure of how well the trained denoising ML model 502′ has learned to make predictions or classifications on new and/or unseen instances. The computing system can receive noisy measured MCG data 508 from a user device (e.g., from the user device 120 through the network 102 as illustrated in FIG. 1) and provide the noisy measured MCG data 508 as input to the trained denoising ML model 502′ running on the computing system. In some implementations, the noisy measured MCG data 508 can be the measured MCG data 131a of FIG. 1 obtained in a target environment. For example, the noisy measured MCG data 508 can be unshielded MCG data generated by the MCG system 122-1 (e.g., an unshielded MCG system) in a magnetically unshielded environment. In some implementations, the noise data 501 and the noisy measured MCG data 508 can be measured in the same target environment. In some implementations, the noise data 501 can be measured in one or more representative environments similar to the target environment where the noisy measured MCG data 508 is measured.

In response, at a processing step 509, the computing system can automatically generate a prediction result 510 by applying the trained denoising ML 502′. The prediction result 510 can include denoised MCG data generated based on the noisy measured MCG data 508. In other words, the computing system can generate the denoised MCG data by removing magnetic noise signals in the noisy measured MCG data 508 using the trained denoising ML model 502′. In some implementations, the trained denoising ML model 502′ can be tailored to noises in the target environment and can improve a signal to noise ratio of the noisy measured MCG data 508.

For example, a clinic can have an unshielded MCG system located in an emergency room (ER). The noise data 501 can have a magnetic noise profile according to an arrangement of the MCG system and the environment in the ER. Before the onset of patient measurement, the unshielded MCG system can be placed in its designated measurement location. The unshielded MCG system can record a background noise internally to determine the noise data 501. The recorded noise data 501 is then added to the simulated MCG data 503 to create the noisy simulated MCG data 504 (e.g., training and/or evaluation inputs). The denoising ML model 502 can undergo the training illustrated above in reference to FIG. 5, and the trained denoising ML model 502′ can be generated for this environment. Once the user initiates a measurement, the noisy measured MCG data 508 is inputted into the trained denoising ML model 502′. The denoised MCG data 510 can be generated for downstream use. In some implementations, if the unshielded MCG system is moved to another location, then such training can be reset and re-iterated.

In some implementations, shielded measured MCG data can be used as the evaluation data 506B. The shielded measured MCG data generated by the MCG system 122-1 (e.g., SQUID) in a magnetically shielded environment. During the evaluation, the shielded measured MCG data can be used as ground truth values and be compared to the predicted result (e.g., the denoised MCG data) of the trained denoising ML model 502′. In some implementations, the metric used to evaluate the performance of the trained denoising ML model 502′ is root mean square error (RMSE) between predicted results and the ground truth values. Based on the result of evaluation (e.g., RMSE), one or more parameters of the trained denoising ML model 502′ can be further adjusted.

In some implementations, one or more parameters of the trained denoising ML model 502′ are updated based on the noisy measured MCG data 508 and the prediction result 510 (e.g., the denoised MCG data). For example, the noisy measured MCG data 508 provided by the user device and the corresponding prediction results can be additionally used as the training data 506A to further train or refine the trained denoising ML model 502′, returning an updated trained denoising ML model 502′. The noisy measured MCG data 508 may contain specific patterns or anomalies relevant to their use case or environment. By incorporating this data into the training process, the trained denoising ML model 502′ can be customized to better suit the needs of users. The diversity of the training data can also be increased.

In some implementations, the computing system takes an action based on the prediction result 510. The computing system can provide the denoised MCG data of the organ of the subject as input of one or more other ML models (e.g., the disease classification ML model 144 or the feature extraction ML model 146 of FIG. 1). For example, the disease classification ML model 144 can automatically generate a prediction result based on the denoised MCG data, and the prediction result can indicate a disease associated with the organ of the subject. In another example, the feature extraction ML model 146 can automatically generate a prediction result based on the denoised MCG data, and the prediction result can indicate a biomarker associated with the organ of the subject.

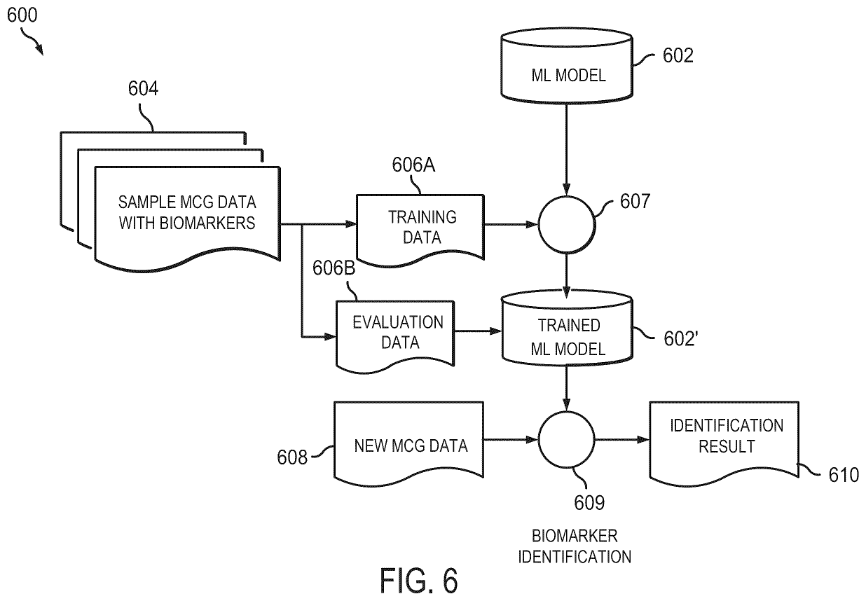

FIG. 6 is a schematic diagram illustrating an example feature extraction process 600 performed by a ML system. The ML system can be an example of the ML system 140 of FIG. 1. The feature extraction process 600 can be implemented by a computing system (e.g., the cloud computing platform 130 of FIG. 1) that includes one or more computing devices and one or more machine-readable repositories or databases that are in communication with each other.

The ML system can include one or more feature extraction ML models 602. The feature extraction ML models 602 can be examples of the feature extraction ML model 146 of FIG. 1. The feature extraction ML models 602 can include a weakly supervised ML model, an unsupervised ML model, or a clustering ML algorithm, or any suitable combination thereof. In some implementations, sample MCG data with biomarkers 604 are prepared for training and/or evaluating the feature extraction ML models 602. In some implementations, the computing system can store the sample MCG data with biomarkers 604 in a repository (e.g., the ECG/MCG database 150 of FIG. 1) accessible by the one or more feature extraction ML models 602. The sample MCG data with biomarkers 604 can be divided into training data 606A and evaluation data 606B. At a processing step 607, the computing system can train one of the feature extraction ML models 602 with respect to the training data 606A and return a trained feature extraction ML model 602′. In some implementations, after training the feature extraction ML model 602 with the training data 606A, the trained feature extraction ML model 602′ is evaluated with the evaluation data 606B. The evaluation data 606B can provide independent measure of how well the trained feature extraction ML model 602′ has learned to make predictions or classifications on new and/or unseen instances. The computing system can receive new MCG data 608 from a user device (e.g., from the user device 120 through the network 102 as illustrated in FIG. 1) and provide the new MCG data 608 as input to the trained feature extraction ML model 602′ running on the computing system. The new MCG data 608 is associated with an organ of a subject. In response, at a processing step 609, the computing system can automatically generate a prediction result 610 by applying the trained feature extraction ML 602′. The prediction result 610 can indicate a biomarker identification result associated with the organ of the subject. For examples, the biomarker identification result can indicate one of predetermined biomarkers associated with the organ of the subject. The predetermined biomarkers include, but are not limited to, an R intervals, a repolarization rate, patient demographical information, an R-T peak angle of the MCG data, or any combination thereof. In some implementations, the prediction result 610 can include one or more new biomarkers that humans may not be able to derive. For example, the new biomarkers can include graphic core features of the MCG data. In some implementations, the computing system takes an action based on the prediction result 610.

The sample MCG data with biomarkers 604 includes simulated MCG data (e.g., the simulated MCG data 137 of FIG. 1) and biomarkers associated with the simulated MCG data. The sample MCG data with biomarkers 604 can further include measured MCG data (e.g., the measured MCG data 131a generated by the MCG system 122-1 of FIG. 1). For example, the sample MCG data with biomarkers 604 can include shielded MCG data generated by the MCG system 122-1 (e.g., SQUID) in a magnetically shielded environment. In some implementations, the sample MCG data with biomarkers 604 can include denoised MCG data generated based on the measured MCG data 131a. For example, the measured MCG data 131a can be unshielded MCG data generated by the MCG system 122-1 (e.g., an unshielded MCG system) in a magnetically unshielded environment. In this case, the denoising model 142 can be used to generate the denoised MCG data by removing magnetic noise signals in the measured MCG data 131a. The denoised MCG data can be included in the sample MCG data with biomarkers 604 for training and/or evaluation purposes.

In some implementations, one or more parameters of the trained feature extraction ML model 602′ are updated based on the new MCG data 608 and the prediction result 610. For example, the new MCG data 608 provided by the user device and the corresponding prediction results can be additionally used as the training data 606A to further train or refine the trained feature extraction ML model 602′, returning an updated trained feature extraction ML model 602′. The new MCG data 608 may contain specific patterns or anomalies relevant to their use case or environment. By incorporating this data into the training process, the trained feature extraction ML model 602′ can be customized to better suit the needs of users. The diversity of the training data can also be increased.

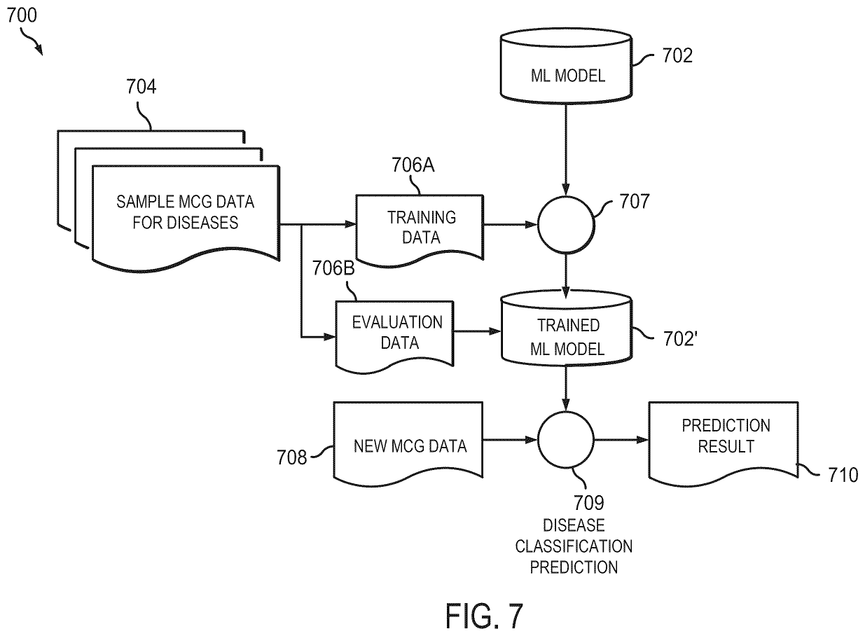

FIG. 7 is a schematic diagram illustrating an example disease classification process 700 performed by a ML system. The ML system can be an example of the ML system 140 of FIG. 1. The disease classification process 700 can be implemented by a computing system (e.g., the cloud computing platform 130 of FIG. 1) that includes one or more computing devices and one or more machine-readable repositories or databases that are in communication with each other.

The ML system can include one or more disease classification ML models 702. The disease classification ML models 702 can be examples of the disease classification ML model 144 of FIG. 1. The disease classification ML models 702 can include at least one of an Extreme Gradient Boosting (XG BOOST) model, a Visual Geometry Group (VGG) model, or a deep learning model. In some implementations, sample MCG data 704 are prepared for training and/or evaluating the disease classification ML models 702. In some implementations, the computing system can store the sample MCG data 704 in a repository (e.g., the ECG/MCG database 150 of FIG. 1) accessible by the one or more disease classification ML models 702. The sample MCG data 704 can be divided into training data 706A and evaluation data 706B. At a processing step 707, the computing system can train one of the disease classification ML models 702 with respect to the training data 706A and return a trained disease classification ML model 702′. In some implementations, after training the disease classification ML model 702 with the training data 706A, the trained disease classification ML model 702′ is evaluated with the evaluation data 706B. The evaluation data 706B can provide independent measure of how well the trained disease classification ML model 702′ has learned to make predictions or classifications on new and/or unseen instances. The computing system can receive new MCG data 708 from a user device (e.g., from the user device 120 through the network 102 as illustrated in FIG. 1) and provide the new MCG data 708 as input to the trained disease classification ML model 702′ running on the computing system. The new MCG data 708 is associated with an organ of a subject. In response, at a processing step 709, the computing system can automatically generate a prediction result 710 by applying the trained disease classification ML 702′. The prediction result 710 can indicate a disease classification result associated with the organ of the subject. For examples, a disease classification result can indicate one of predetermined diseases or conditions associated with the organ of the subject. The predetermined diseases or conditions include, but are not limited to, ischemia, cardiomyocyte injury, myocardial infarction, left ventricular hypertrophy, or any combination thereof. In some implementations, the prediction result 710 can further include a confidence level, indicating a likelihood of the organ of the subject being associated with one of the predetermined diseases or conditions. In some implementations, the computing system takes an action based on the prediction result 710.

The sample MCG data 704 includes simulated MCG data (e.g., the simulated MCG data 137 of FIG. 1). The sample MCG data 704 can further include measured MCG data (e.g., the measured MCG data 131a generated by the MCG system 122-1 of FIG. 1). For example, the sample MCG data 704 can include shielded MCG data generated by the MCG system 122-1 (e.g., SQUID) in a magnetically shielded environment. In some implementations, the sample MCG data 704 can include denoised MCG data generated based on the measured MCG data 131a. For example, the measured MCG data 131a can be unshielded MCG data generated by the MCG system 122-1 (e.g., an unshielded MCG system) in a magnetically unshielded environment. In this case, the denoising model 142 can be used to generate the denoised MCG data by removing magnetic noise signals in the measured MCG data 131a. The denoised MCG data can be included in the sample MCG data 704 for training and/or evaluation purposes.

In some implementations, the simulated MCG data can be used as the training data 706A, and the measured MCG data can be used as the evaluation data 706B. For example, during the evaluation (also referred to as refined training), the predicted result of the trained disease classification ML model 702′ using the evaluation data 706B can be compared to ground truth values. In some implementations, the metric used to evaluate the performance of the trained disease classification ML model 702′ is root mean square error (RMSE) between predicted results and the ground truth values. Based on the result of evaluation (e.g., RMSE), one or more parameters of the trained disease classification ML model 702′ can be further adjusted.

In some implementations, one or more parameters of the trained disease classification ML model 702′ are updated based on the new MCG data 708 and the prediction result 710. For example, the new MCG data 708 provided by the user device and the corresponding prediction results can be additionally used as the training data 706A to further train or refine the trained disease classification ML model 702′, returning an updated trained disease classification ML model 702′. The new MCG data 708 may contain specific patterns or anomalies relevant to their use case or environment. By incorporating this data into the training process, the trained disease classification ML model 702′ can be customized to better suit the needs of users. The diversity of the training data can also be increased.

In some implementations, the disease classification ML model 702 can be an XG BOOST model or a VGG model. In this case, biomarkers of the sample MCG data 704 can be used by the computing system to train the disease classification ML model 702 at the processing step 707. In one example, the biomarkers can include an R interval, a repolarization rate, and patient demographical information associated with the sample MCG data 704. In another example, the biomarkers can include a R-T peak angle of the sample MCG data 704. In another example, the biomarkers can include graphic core features of the sample MCG data 704 that are generated by the feature extraction model 146. In another example, the biomarkers can be a combination of any of the above-mentioned features. Accordingly, biomarkers of the new MCG data 708 can be used by the computing system to generate the prediction result 710 at the processing step 709.

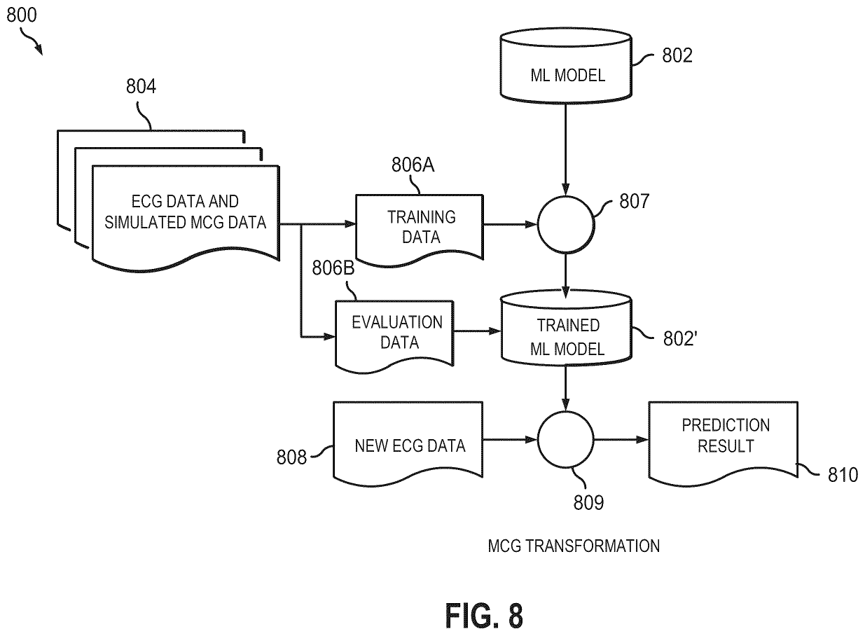

FIG. 8 is a schematic diagram illustrating an example MCG transformation process 800 performed by a ML system. The ML system can be an example of the ML system 140 of FIG. 1. The MCG transformation process 800 can be implemented by a computing system (e.g., the cloud computing platform 130 of FIG. 1) that includes one or more computing devices and one or more machine-readable repositories or databases that are in communication with each other.

The ML system can include one or more MCG transformation ML models 802. The MCG transformation ML models 802 can be examples of the MCG transformation ML model 148 of FIG. 1. In some implementations, the MCG transformation ML models 802 can include a neural network. In some implementations, sample ECG/MCG data 804 are prepared for training and/or evaluating the MCG transformation ML models 802. In some implementations, the computing system can store the sample ECG/MCG data 804 in a repository (e.g., the ECG/MCG database 150 of FIG. 1) accessible by the one or more MCG transformation ML models 802. The sample ECG/MCG data 804 can be divided into training data 806A and evaluation data 806B. At a processing step 807, the computing system can train one of the MCG transformation ML models 802 with respect to the training data 806A and return a trained MCG transformation ML model 802′. In some implementations, after training the MCG transformation ML model 802 with the training data 806A, the trained MCG transformation ML model 802′ is evaluated with the evaluation data 806B. The evaluation data 806B can provide independent measure of how well the trained MCG transformation ML model 802′ has learned to make predictions or classifications on new and/or unseen instances. The computing system can receive new ECG data 808 from a user device (e.g., from the user device 120 through the network 102 as illustrated in FIG. 1) and provide the new ECG data 808 as input to the trained MCG transformation ML model 802′ running on the computing system. The new ECG data 808 is associated with an organ of a subject. In response, at a processing step 809, the computing system can automatically generate a prediction result 810 by applying the trained MCG transformation ML 802′. The prediction result 810 can include MCG data associated with the organ of the subject. In some implementations, the computing system takes an action based on the prediction result 810.

The sample ECG/MCG data 804 can include ECG data and simulated MCG data generated based on the ECG data. In some implementations, the ECG data can include the ECG data 133a (e.g., 12-lead ECG data) of FIG. 1, and the simulated MCG data can include the simulated MCG data 137 generated based on the ECG data 133a (e.g., using the VCG engine 134 and the MCG engine 136 as described above in reference to FIG. 1).

In some implementations, the sample ECG/MCG data 804 can include the ECG data 133a and the measured MCG data 131a. For example, the ECG data 133a can be associated with an organ of a subject, and the measured MCG data 131a can be MCG data of the organ of the same subject. In some implementations, the measured MCG data 131a can be representative data of the same organ but from different subject(s). The ECG data 133a and the measured MCG data 131a can be included in the evaluation data 806B. During the evaluation, the computing system can apply the trained MCG transformation ML model 802′ to the ECG data 133a and generate simulated MCG data. The computing system can compare the simulated MCG data with the measured MCG data 131a to obtain a comparison result. The comparison result can be used to improve the trained MCG transformation ML model 802′. In some implementations, the comparison can be based on a biomarker of the simulated MCG data and the measured MCG data 131a. For example, the computing system can evaluate whether the simulated MCG data and the measured MCG data 131a have similar T-wave field max angles. Parameters used by the trained MCG transformation ML model 802′ can be adjusted based on the comparison result.

In some implementations, one or more parameters of the trained MCG transformation ML model 802′ are updated based on the new ECG data 808 and the prediction result 810. For example, the new ECG data 708 provided by the user device and the corresponding prediction results can be additionally used as the training data 806A to further train or refine the trained MCG transformation ML model 802′, returning an updated trained MCG transformation ML model 802′. The new ECG data 808 may contain specific patterns or anomalies relevant to their use case or environment. By incorporating this data into the training process, the trained disease classification ML model 802′ can be customized to better suit the needs of users. The diversity of the training data can also be increased.

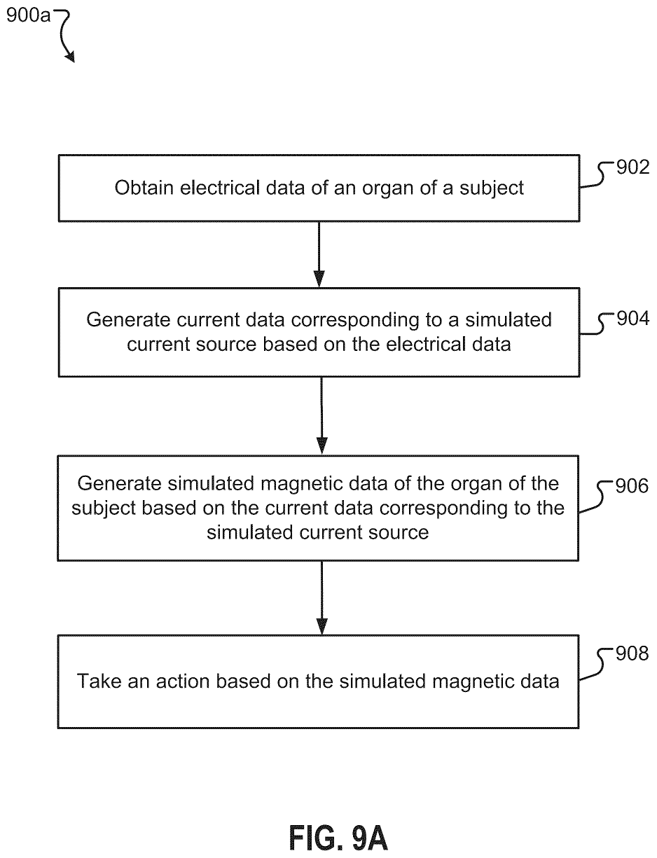

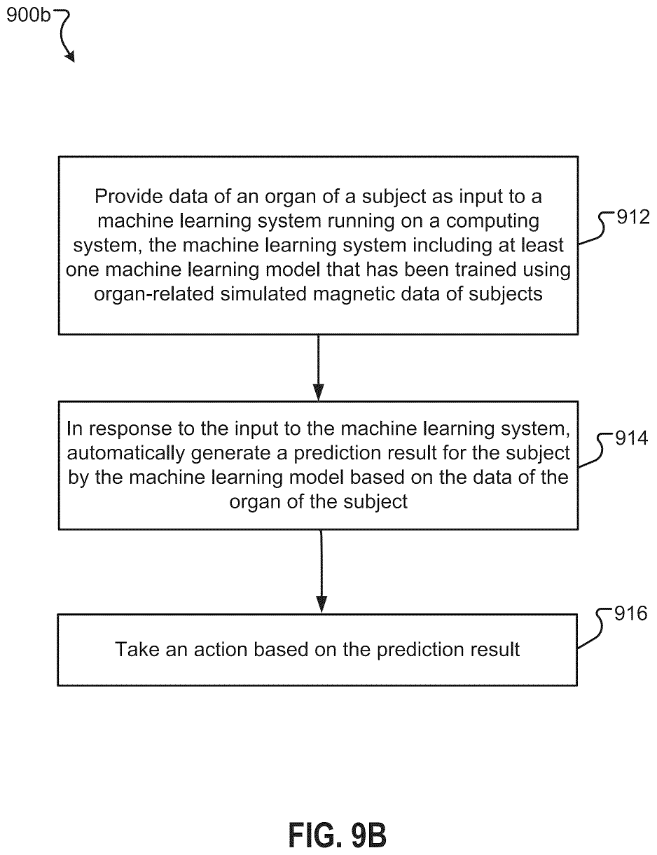

FIG. 9A illustrates a flow chart of an example process 900a of generating simulated magnetic data. The process 900a can be performed by any suitable computing system (e.g., the cloud computing platform 130 of FIG. 1).

The process 900a can begin at operation 902, where electrical data (e.g., the ECG data 133a or the VCG data 135 of FIG. 1) of an organ of a subject is obtained. In some implementations, obtaining the electrical data of the organ of the subject can include transforming measured current data (e.g., the ECG data 133a) of the organ of the subject into the electrical data (e.g., the VCG data 135) using a transformation approach. The transformation approach can include at least one of Kors Regression, Kors quasi-orthogonal, inverse Dower, or a linear regression-based transformation.

In some implementations, the electrical data includes vectorcardiography (VCG) data (e.g., the VCG data 135), and the simulated magnetic data includes MCG data (e.g., the simulated MCG data 137). The process 900a can further include receiving electrocardiography (ECG) data of the organ of the subject and converting the ECG data to VCG data.

At operation 904, current data corresponding to a current source based on the electrical data of the organ of the subject is generated. The current source can be independent from a physiological geometry of the organ of the subject. In some implementations, the electrical data can include 12-lead ECG measurement data (e.g., the ECG data 133a). The 12-lead ECG measured data can include 1×12 matrix data, and the current source can include 1×3 matrix data.

In some implementations, the current source is derived by approximating an electrical current of the organ of the subject as a set of current dipoles (e.g., as described in the operation 208 of FIG. 2A). An electrical activity of the organ at a single time point can be represented by a corresponding single current dipole. The current data can include information of an average direction of a current flowing in the organ of the subject at a given point in time.

In some implementations, generating the current data corresponding to the current source based on the electrical data of the organ of the subject includes: for each single time point of a plurality of sample time points corresponding to the electric data, a) extracting corresponding vector amplitudes along axes of a coordinate system of a vector representing the electric data at the single time point (e.g., as described in the operation 206 of FIG. 2A); and b) calculating an electric field value at a given sensor location relative to the current source in the coordinate system (e.g., as described in the operation 208 of FIG. 2A). The current data can include a plurality of electric field values respectively calculated at the plurality of sample time points (e.g., as described in the operation 210 of FIG. 2A).

At operation 906, simulated magnetic data (e.g., the simulated MCG data 137) of the organ of the subject can be generated based on the current data corresponding to the current source. In some implementations, the current source can include a single current dipole. In some other implementations, the current source can be a series of current dipoles. The simulated magnetic data of the organ of the subject can be associated with a synthetic result of magnetic fields from the series of current dipoles of the current source.

In some implementations, generating the simulated magnetic data of the organ of the subject based on the current data includes generating representative data of a magnetic field of the organ of the subject based on the current data by physics-based forward modeling (e.g., as described in the operation 208 of FIG. 2A). The simulated magnetic data can include the representative data of the magnetic field.

In some implementations, generating the simulated magnetic data of the organ of the subject based on the current data includes converting the current data into the simulated magnetic data using a Biot-Savart equation expressed as:

$$B ( r ) = \frac{{μ}_{0}}{4 π} {\int }_{C} \frac{Idl \times {r}^{′}}{{\vert {r}^{′} \vert}^{3}} ,$$

where μ₀ is permeability of free space, r represents a radius away from the current source, B(r) is a magnetic field generated by a given current at radius r away from the current source, dl represents an infinitesimal section of the current source, I is a current magnitude of that section of the current source, C is the path of the current source.

In some implementations, the simulated magnetic data includes data corresponding to a respective magnetic field at a position of each of a plurality of simulated sensors (e.g., the simulated sensors 304 of FIG. 3D or the simulated sensors 406 of FIGS. 4A-4B) near or around the current source.

In some implementations, generating the simulated magnetic data of the organ of the subject includes generating magnetic data at positions between the positions of the plurality of simulated sensors based on the respective magnetic fields at the positions of the plurality of simulated sensors (e.g., the interpolation methods as described with reference to FIG. 3D).

At operation 908, an action based on the simulated magnetic data can be taken. In some implementations, taking the action based on the simulated magnetic data includes presenting a graphical interface (e.g., as shown in FIGS. 4A-4B) corresponding to the simulated magnetic data of the organ of the subject. The simulated magnetic data can include data corresponding to a respective magnetic field at a corresponding position of each of a plurality of simulated sensors near or around the current source. The graphical interface can include at least one of: a magnetic field map (MFM) (e.g., the MFM 408 of FIG. 4A or the MFM 418 of FIG. 4B) at a time point, a trace of the data (e.g., the group of magnetic field traces 404 of FIG. 4A or the group of magnetic field traces 416 of FIG. 4B) corresponding to the respective magnetic fields over a period of time, or a trace of the current data (e.g., the ECG data 133a) corresponding to the current source over the period of time. The magnetic field map can include representative points of the plurality of simulated sensors at the corresponding positions near or around the current source, and an overlay or an average of the respective magnetic fields at the corresponding positions at the time point.

In some implementations, the service provider system 110 can process the simulated or measured magnetic-field values to generate gradiometric magnetic-field measurements. In some examples, the MCG engine 136 can configure a gradiometer using two or more sensors positioned at known spatial offsets and can calculate a first-order or second-order magnetic-field gradient by subtracting the magnetic-field signals between those sensors. The MCG system 120 can then use these gradiometric measurements to suppress common-mode environmental noise and to enhance detection of localized cardiac magnetic sources. When comparing simulated magnetic fields to experimentally acquired data, the service provider system 110 can apply corresponding gradiometric transformations, such that both simulated and measured data are represented in the same form.

In some implementations, taking the action based on the simulated magnetic data includes: comparing the simulated magnetic data (e.g., the simulated MCG data 137) of the organ of the subject with measured magnetic data (e.g., the measured MCG data 131a) to obtain a comparison result; and re-generating the simulated magnetic data of the organ based on the electrical data of the organ of the subject and the comparison result.

In some implementations, the organ is a heart. The current data can include vector data (e.g., the ECG data 133a) that represents electrical current data of the heart. In some implementations, the process 800 further includes generating a set of vector data for each of a plurality of heart beats of the subject.

In some implementations, the process 900a further includes: generating simulated magnetic data (e.g., the simulated MCG data 137) of organs of subjects based on electrical data of the organs of the subjects; and storing the generated simulated magnetic data of the organs of the subjects in a database (e.g., the ECG/MCG database 150 of FIG. 1). The database can include corresponding magnetic data associated with respective organ-related diseases or organ-related biomarkers.

In some implementations, taking the action based on the simulated magnetic data includes: comparing the simulated magnetic data (e.g., the MFM 408 of FIG. 4A) of the organ of the subject with the corresponding magnetic data (e.g., the MFM 418 of FIG. 4B) associated with respective organ-related diseases or organ-related biomarkers; and determining whether the organ of the subject is associated with a specific disease (e.g., left ventricular hypertrophy (LVH) as discussed with reference to FIG. 4B) or a specific biomarker based on a result of the comparing (e.g., as described with reference to FIG. 4B).

FIG. 9B illustrates a flow chart of an example process 900b of using a ML system trained with simulated magnetic data. The process 900b can be performed by any suitable computing system (e.g., the cloud computing platform 130 of FIG. 1).

The process 900b can begin at operation 912, where the computing system provides data of an organ of a subject (e.g., the noisy measured MCG data 508 of FIG. 5, the new MCG data 608 of FIG. 6, the new MCG data 708 of FIG. 7, or the new ECG data 808 of FIG. 8) as input to a machine learning system (e.g., the ML system 140 of FIG. 1) running on a computing system. The machine learning system includes at least one machine learning model (e.g., the trained denoising ML model 502′, the trained feature extraction ML model 602′, the trained disease classification ML model 702′, or the trained MCG transformation ML model 802′) that has been trained using organ-related simulated magnetic data (e.g., the simulated MCG data 503, the sample MCG data with biomarkers 604, the sample MCG data 704, or the ECG data and simulated MCG data 804) of subjects.

In some implementations, the organ-related simulated magnetic data of the subjects are generated based on organ-related electrical data of the subjects (e.g., the ECG data 133a or the VCG data 135 of FIG. 1). For example, the computing system can generate, for each piece of organ-related electric data, current data corresponding to a simulated electrical dipole source based on the piece of organ-related electrical data (e.g., as described in the operation 208 of FIG. 2A). The computing system can generate corresponding organ-related simulated magnetic data (e.g., the simulated MCG data 137) based on the current data corresponding to the simulated electrical dipole source. In some implementations, the simulated electrical dipole source is independent from a physiological geometry of the organ.

At operation 914, in response to the input to the machine learning system, the computing system automatically generates a prediction result for the subject by the machine learning model based on the data of the organ of the subject.

In some implementations, the organ-related simulated magnetic data of the subjects includes corresponding magnetic data associated with respective organ-related diseases (e.g., ischemia, cardiomyocyte injury, myocardial infarction, left ventricular hypertrophy, or any combination thereof). The at least one machine learning model includes an organ-related disease classification model (e.g., the trained disease classification ML models 702′ of FIG. 7). The prediction result (e.g., the prediction result 710) indicates a disease classification result associated with the organ of the subject.

In some implementations, the organ-related simulated magnetic data of the subjects includes corresponding magnetic data associated with respective organ-related biomarkers (e.g., an R interval, a repolarization rate, patient demographical information, an R-T peak angle of the MCG data, graphic core features of the MCG data, or any combination thereof). The at least one machine learning model includes an organ-related biomarker identification model (e.g., the trained feature extraction ML 602′ of FIG. 6). The prediction result (e.g., the prediction result 610) indicates a biomarker identification result associated with the organ of the subject.

In some implementations, the data of the organ of the subject includes magnetic data of the organ measured with an unshielded magnetic sensing device (e.g., the unshielded MCG system 122-1 of FIG. 1). The at least one machine learning model includes a denoising model (e.g., the trained denoising ML model 502′ of FIG. 5) configured to denoise the magnetic data measured with the unshielded magnetic sensing device.

In some implementations, the computing system can train the denoising model by generating noisy magnetic data (e.g., the noisy simulated MCG data 504) based on noise data (e.g., the noise data 501) and the organ-related simulated magnetic data of the subjects (e.g., the simulated MCG data 503). The noise data is associated with a measurement environment where the magnetic data of the organ is measured with the unshielded magnetic sensing device. The computing system can train the denoising model with the noisy magnetic data as the input of the denoising model and the organ-related simulated magnetic data of the subjects as output of the denoising model. In some implementations, the denoising model includes a denoising autoencoder.

At operation 916, the computing system takes an action based on the prediction result. In some implementations, the prediction result (e.g., the prediction result 510 of FIG. 5) can include denoised MCG data generated based on noisy measured MCG data (e.g., the noisy measured MCG data 508). In some implementations, the organ-related simulated magnetic data of the subjects includes corresponding magnetic data associated with respective organ-related diseases or organ-related biomarkers. The at least one machine learning model includes at least one of an organ-related disease classification model (e.g., the trained disease classification ML model 702′ of FIG. 7) or an organ-related biomarker identification model (e.g., the trained feature extraction ML 602′ of FIG. 6). In some implementations, the computing system can provide the denoised magnetic data outputted by the denoising model as input of the trained disease classification ML model 702′. The computing system can automatically generate a prediction result by the trained disease classification ML model 702′. The prediction result generated by the trained disease classification ML model 702′ indicates a disease classification result associated with the organ of the subject. In some other implementations, the computing system can provide the denoised magnetic data outputted by the denoising model as input of the trained feature extraction ML 602′. The computing system can automatically generate a prediction result by the trained feature extraction ML 602′. The prediction result generated by the trained feature extraction ML 602′ indicates a biomarker identification result associated with the organ of the subject.

In some implementations, the process 900b further includes receiving measured electrical data of the organ of the subject (e.g., the ECG data 133a of FIG. 1). The computing system can generate current data corresponding to a simulated electrical dipole source based on the electrical data of the organ of the subject (e.g., as described in the operation 208 of FIG. 2A). The computing system can generate simulated magnetic data of the organ of the subject (e.g., the simulated MCG data 137) based on the current data corresponding to the simulated electrical dipole source. The data of the organ of the subject provided as the input to the machine learning system includes the generated simulated magnetic data of the organ of the subject.

In some implementations, the organ-related simulated magnetic data of subjects is stored in a repository (e.g., the ECG/MCG database 150 of FIG. 1) accessible by the at least one machine learning model. The process 900b further includes storing the generated simulated magnetic data of the organ of the subject (e.g., the simulated MCG data 137) and the prediction result in the repository. The process 900b further includes training the at least one machine learning model based on organ-related magnetic data (e.g., MCG data stored in the ECG/MCG data base 150) in the repository including the generated simulated magnetic data of the organ of the subject (e.g., the simulated MCG data 137).

In some implementations, the data of the organ of the subject includes ECG data of the organ of the subject. The at least one machine learning model includes an MCG transformation model (e.g., the trained MCG transformation ML model 802′ of FIG. 8) that has been trained using a plurality of ECG datasets (e.g., the ECG data 133a of FIG. 1) and a plurality of corresponding simulated MCG datasets (e.g., the simulated MCG data 137 of FIG. 1) based on the plurality of ECG datasets. The prediction result for the subject includes a corresponding simulated MCG dataset for the subject derived by the MCG transformation model based on the ECG data of the organ of the subject.

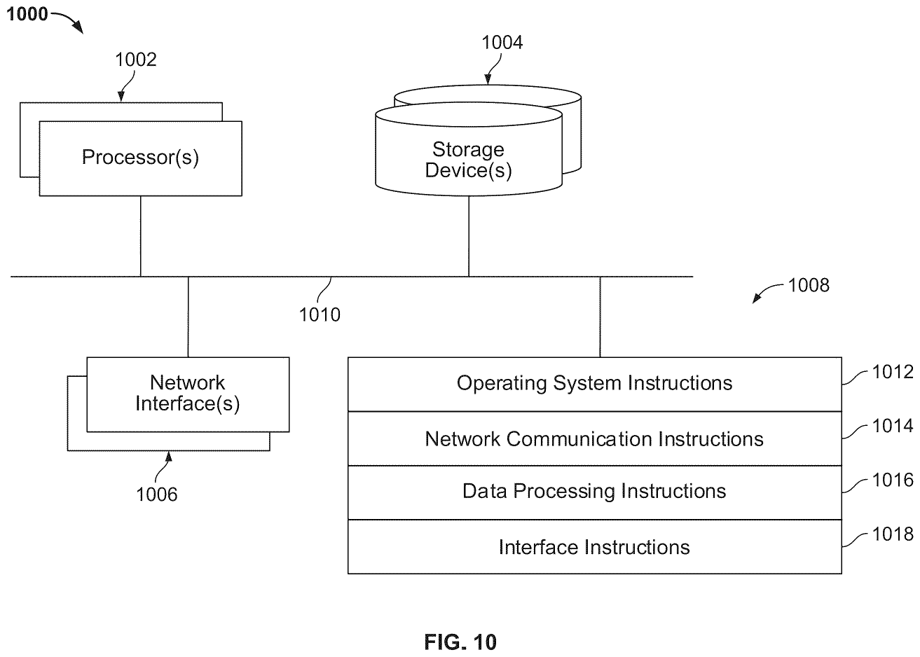

FIG. 10 is an example architecture 1000 for a cloud computing system (e.g., the cloud computing platform 130 described in reference to FIG. 1). Other architectures are possible, including architectures with more or fewer components. In some implementations, architecture 1000 includes one or more processor(s) 1002 (e.g., dual-core Intel® Xeon® Processors), one or more network interface(s) 1006, one or more storage device(s) 1004 (e.g., hard disk, optical disk, flash memory) and one or more computer-readable medium(s) 1008 (e.g., hard disk, optical disk, flash memory, etc.). These components can exchange communications and data over one or more communication channel(s) 1010 (e.g., buses), which can utilize various hardware and software for facilitating the transfer of data and control signals between components.

The term “computer-readable medium” refers to any medium that participates in providing instructions to the processor(s) 1002 for execution, including without limitation, non-volatile media (e.g., optical or magnetic disks), volatile media (e.g., memory) and transmission media. Transmission media includes, without limitation, coaxial cables, copper wire, and fiber optics.

Computer-readable medium(s) 1008 can further include instructions 1012 for an operating system (e.g., Mac OS® server, Windows® NT server, Linux Server), instructions 1014 for network communications module, data processing instructions 1016, and interface instructions 1018.

Operating systems can be multi-user, multiprocessing, multitasking, multithreading, real time, etc. Operating system performs basic tasks, including but not limited to: recognizing input from and providing output to devices 1002, 1004, 1006 and 1008; keeping track and managing files and directories on computer-readable medium(s) 1008 (e.g., memory or a storage device); controlling peripheral devices; and managing traffic on the one or more communication channel(s) 1010. Network communications module includes various components for establishing and maintaining network connections (e.g., software for implementing communication protocols, such as TCP/IP, HTTP, etc.) and for creating a distributed streaming platform using, for example, Apache Kafka™. Data processing instructions 1016 include server-side or backend software for implementing the server-side operations. Interface instructions 1018 includes software for implementing a web server and/or portal for sending and receiving data to and from user side computing devices and service side computing devices.

Architecture 1000 can be implemented by a cloud computing system and can be included in any computer device, including one or more server computers in a local or distributed network each having one or more processing cores. Architecture 1000 can be implemented in a parallel processing or peer-to-peer infrastructure or on a single device with one or more processors. Software can include multiple software components or can be a single body of code.

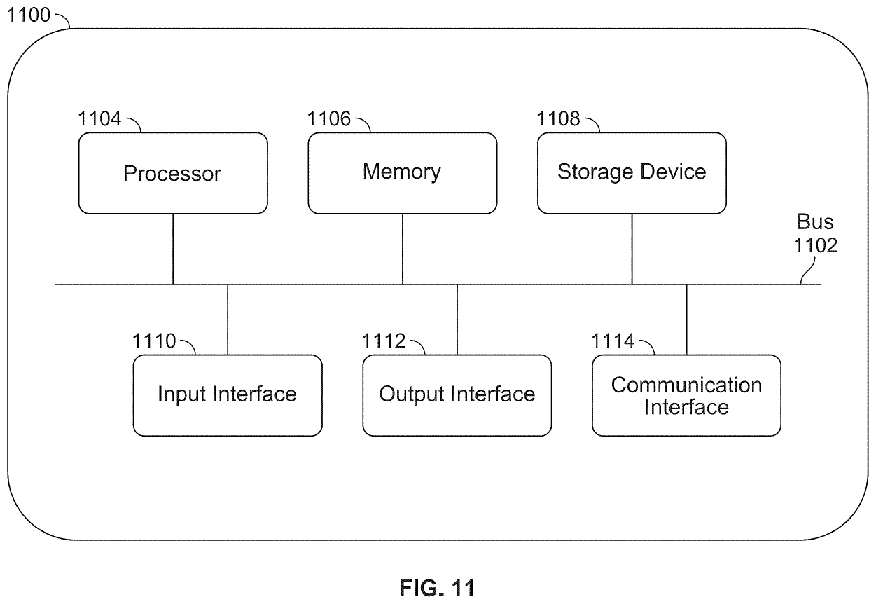

FIG. 11 illustrates an example architecture for a computing device (e.g., the service provider system 110 or any of the user devices 120 of FIG. 1). Referring now to FIG. 11, illustrated is a schematic diagram of a device 1100. Device 1100 includes processor 1104, memory 1106, storage component 1108, input interface 1110, output interface 1112, communication interface 1114, and bus 1102.

Bus 1102 includes a component that permits communication among the components of device 1100. In some embodiments, processor 1104 is implemented in hardware, software, or a combination of hardware and software. In some examples, processor 1104 includes a processor (e.g., a central processing unit (CPU), a graphics processing unit (GPU), an accelerated processing unit (APU), and/or the like), a microphone, a digital signal processor (DSP), and/or any processing component (e.g., a field-programmable gate array (FPGA), an application specific integrated circuit (ASIC), and/or the like) that can be programmed to perform at least one function. Memory 1106 includes random access memory (RAM), read-only memory (ROM), and/or another type of dynamic and/or static storage device (e.g., flash memory, magnetic memory, optical memory, and/or the like) that stores data and/or instructions for use by processor 1104.

Storage component 1108 stores data and/or software related to the operation and use of device 1100. In some examples, storage component 1108 includes a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, a CD-ROM, RAM, PROM, EPROM, FLASH-EPROM, NV-RAM, and/or another type of computer readable medium, along with a corresponding drive.

Input interface 1110 includes a component that permits device 1100 to receive information, such as via user input (e.g., a touchscreen display, a keyboard, a keypad, a mouse, a button, a switch, a microphone, a camera, and/or the like). Additionally or alternatively, in some embodiments input interface 1110 includes a sensor that senses information (e.g., a global positioning system (GPS) receiver, an accelerometer, a gyroscope, an actuator, and/or the like). Output interface 1112 includes a component that provides output information from device 1100 (e.g., a display, a speaker, one or more light-emitting diodes (LEDs), and/or the like).

In some embodiments, communication interface 1114 includes a transceiver-like component (e.g., a transceiver, a separate receiver and transmitter, and/or the like) that permits device 1100 to communicate with other devices via a wired connection, a wireless connection, or a combination of wired and wireless connections. In some examples, communication interface 1114 permits device 1100 to receive information from another device and/or provide information to another device. In some examples, communication interface 1114 includes an Ethernet interface, an optical interface, a coaxial interface, an infrared interface, a radio frequency (RF) interface, a universal serial bus (USB) interface, a Wi-Fi® interface, a cellular network interface, and/or the like.

In some embodiments, device 1100 performs one or more processes described herein. Device 1100 performs these processes based on processor 1104 executing software instructions stored by a computer-readable medium, such as memory 1106 and/or storage component 1108. A computer-readable medium (e.g., a non-transitory computer readable medium) is defined herein as a non-transitory memory device. A non-transitory memory device includes memory space located inside a single physical storage device or memory space spread across multiple physical storage devices.

In some embodiments, software instructions are read into memory 1106 and/or storage component 1108 from another computer-readable medium or another device via communication interface 1114. When executed, software instructions stored in memory 1106 and/or storage component 1108 cause processor 1104 to perform one or more processes described herein. Additionally or alternatively, hardwired circuitry is used in place of or in combination with software instructions to perform one or more processes described herein. Thus, embodiments described herein are not limited to any specific combination of hardware circuitry and software unless explicitly stated otherwise.

Memory 1106 and/or storage component 1108 includes data storage or at least one data structure (e.g., a database and/or the like). Device 1100 is capable of receiving information from, storing information in, communicating information to, or searching information stored in the data storage or the at least one data structure in memory 1106 or storage component 1108. In some examples, the information includes network data, input data, output data, or any combination thereof.

In some embodiments, device 1100 is configured to execute software instructions that are either stored in memory 1106 and/or in the memory of another device (e.g., another device that is the same as or similar to device 1100). As used herein, the term “module” refers to at least one instruction stored in memory 1106 and/or in the memory of another device that, when executed by processor 1104 and/or by a processor of another device (e.g., another device that is the same as or similar to device 1100) cause device 1100 (e.g., at least one component of device 1100) to perform one or more processes described herein. In some embodiments, a module is implemented in software, firmware, hardware, and/or the like.

The number and arrangement of components illustrated in FIG. 11 are provided as an example. In some embodiments, device 1100 can include additional components, fewer components, different components, or differently arranged components than those illustrated in FIG. 11. Additionally or alternatively, a set of components (e.g., one or more components) of device 1100 can perform one or more functions described as being performed by another component or another set of components of device 1100.

Embodiments of the subject matter and the functional operations described in the present disclosure can be implemented in digital electronic circuitry, in tangibly-embodied computer software or firmware, in computer hardware, including the structures disclosed in the present disclosure and their structural equivalents, or in combinations of one or more of them. Embodiments of the subject matter described in the present disclosure can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in the present disclosure can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated into special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in the present disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in the present disclosure can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in the present disclosure, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While the present disclosure contains many specific implementation details, these should not be construed as limitations on the scope of any invention or on the scope of what may be claimed, but rather as descriptions of features that may be specific to particular embodiments of particular inventions. Certain features that are described in the present disclosure in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method comprising:
obtaining, by one or more processors, electrical data of an organ of a subject;
generating, by the one or more processors, current data corresponding to a current source based on the electrical data of the organ of the subject;
generating, by the one or more processors, simulated magnetic data of the organ of the subject based on the current data corresponding to the current source; and
taking an action based on the simulated magnetic data.

2. The method of claim 1, wherein the current source is independent from a physiological geometry of the organ of the subject.

3. The method of claim 1, wherein the current source is derived by approximating an electrical current of the organ of the subject as a set of current dipoles, and an electrical activity of the organ at a single time point is represented by a corresponding single current dipole.

4. The method of claim 1, wherein the organ is a heart, and wherein the current data comprises vector data that represents electrical current data of the heart, and
wherein the method further comprises:
generating a set of vector data for each of a plurality of heart beats of the subject.

5. The method of claim 1, wherein the current data comprises information of an average direction of a current flowing in the organ of the subject at a given point in time.

6. The method of claim 1, wherein the current source comprises a single current dipole.

7. The method of claim 1, wherein the current source comprises a series of current dipoles, and
wherein the simulated magnetic data of the organ of the subject is associated with a synthetic result of magnetic fields from the series of current dipoles of the current source.

8. The method of claim 1, wherein generating the simulated magnetic data of the organ of the subject based on the current data comprises:
generating representative data of a magnetic field of the organ of the subject based on the current data by physics-based forward modeling, wherein the simulated magnetic data comprises the representative data of the magnetic field.

9. The method of claim 1, wherein generating the simulated magnetic data of the organ of the subject based on the current data comprises:
converting the current data into the simulated magnetic data using a Biot-Savart equation by integrating the current data along a path C of the current source according to the Biot-Savart equation expressed as:
$$B ( r ) = \frac{{μ}_{0}}{4 π} {\int }_{C} \frac{Idl \times {r}^{′}}{{\vert {r}^{′} \vert}^{3}}$$
where μ₀ is permeability of free space, r represents a radius away from the current source, B(r) is a magnetic field generated by a given current at radius r away from the current source, dl represents an infinitesimal section of the current source, I is a current magnitude of that section of the current source, C is the path of the current source.

10. The method of claim 9, wherein the path of the current source is approximated as a straight wire with a length.

11. The method of claim 1, wherein obtaining the electrical data of the organ of the subject comprises:
transforming measured current data of the organ of the subject into the electrical data using a transformation approach,
wherein the transformation approach comprises at least one of Kors Regression, Kors quasi-orthogonal, inverse Dower, or a linear regression-based transformation.

12. The method of claim 1, wherein generating the current data corresponding to the current source based on the electrical data of the organ of the subject comprises:
for each single time point of a plurality of sample time points corresponding to the electric data,
extracting corresponding vector amplitudes along axes of a coordinate system of a vector representing the electric data at the single time point; and
calculating an electric field value at a given sensor location relative to the current source in the coordinate system,
wherein the current data comprises a plurality of electric field values respectively calculated at the plurality of sample time points.

13. The method of claim 1, wherein the electrical data comprises vectorcardiography (VCG) data, and the simulated magnetic data comprises MCG data, and
wherein the method comprises:
receiving electrocardiography (ECG) data of the organ of the subject; and
converting the ECG data to VCG data.

14. The method of claim 13, wherein the electrical data comprises 12-lead ECG measurement data, and
wherein the 12-lead ECG measured data comprises 1×12 matrix data, and the current source comprises 1×3 matrix data.

15. The method of claim 1, wherein the simulated magnetic data comprises data corresponding to a respective magnetic field at a position of each of a plurality of simulated sensors near or around the current source.

16. The method of claim 15, wherein generating the simulated magnetic data of the organ of the subject comprises:
generating magnetic data at positions between the positions of the plurality of simulated sensors based on the respective magnetic fields at the positions of the plurality of simulated sensors.

17. The method of claim 1, wherein taking the action based on the simulated magnetic data comprises:
forwarding the simulated magnetic data of the organ of the subject for display on a graphical interface, wherein the simulated magnetic data comprises data corresponding to a respective magnetic field at a corresponding position of each of a plurality of simulated sensors near or around the current source, wherein the simulated magnetic data further comprises gradiometric magnetic-field data obtained by subtracting magnetic-field signals between two or more of the plurality of simulated sensors to compute a magnetic-field gradient, and
wherein the simulated magnetic data is configured to display via the graphical interface comprising at least one of:
a magnetic field map at a time point, the magnetic field map comprising representative points of the plurality of simulated sensors at the corresponding positions near or around the current source, and an overlay or an average of the respective magnetic fields at the corresponding positions at the time point,
a trace of the data corresponding to the respective magnetic fields over a period of time, or
a trace of the current data corresponding to the current source over the period of time.

18. The method of claim 1, wherein taking the action based on the simulated magnetic data comprises:
comparing the simulated magnetic data of the organ of the subject with measured magnetic data to obtain a comparison result, wherein the measured magnetic data can be at least one of data measured on the same subject or representative data measured on other subjects; and
re-generating the simulated magnetic data of the organ based on the electrical data of the organ of the subject and the comparison result.

19. The method of claim 1, comprising:
generating, by the one or more processors, simulated magnetic data of organs of subjects based on electrical data of the organs of the subjects; and
storing, by the one or more processors, the generated simulated magnetic data of the organs of the subjects in a database.

20. The method of claim 19, wherein the database comprises corresponding magnetic data associated with respective organ-related diseases or organ-related biomarkers, and
wherein taking the action based on the simulated magnetic data comprises:
comparing the simulated magnetic data of the organ of the subject with the corresponding magnetic data associated with respective organ-related diseases or organ-related biomarkers; and
alerting a relevant party association between the organ of the subject and a specific disease or a specific biomarker based on a result of the comparing.

21. A method comprising:
receiving, by one or more processors, vectorcardiography (VCG) data for a subject;
generating, by the one or more processors, current data corresponding to a current source based on the vectorcardiography (VCG) data;
generating, by the one or more processors, simulated magnetocardiography (MCG) data for the subject based on the current data corresponding to the current source; and
taking an action based on the simulated magnetocardiography (MCG) data.

22. A system comprising:
one or more computers; and
one or more storage devices on which are stored instructions that are operable, when executed by the one or more computers, to cause the one or more computers to perform operations comprising:
obtaining, by one or more processors, electrical data of an organ of a subject;
generating, by the one or more processors, current data corresponding to a current source based on the electrical data of the organ of the subject;
generating, by the one or more processors, simulated magnetic data of the organ of the subject based on the current data corresponding to the current source; and
taking an action based on the simulated magnetic data.
